(19) United States
(12) Patent Application Publication
(10) Pub. No.: US 2024/0035072 A1
(43) Pub. Date: Feb. 1, 2024

(54) TADF EMITTERS FOR IN SITU DETECTION AND REDUCTION OF AUTOFLUORESCENCE

(71) Applicant: 10x Genomics, Inc., Pleasanton, CA (US)

(72) Inventor: Cheyenne CHRISTOPHERSON, San Ramon, CA (US)

(21) Appl. No.: 18/360,137

(22) Filed: Jul. 27, 2023

Related U.S. Application Data
(60) Provisional application No. 63/393,022, filed on Jul. 28, 2022.

(51) Int. Cl. C12Q 1/6841; G01N 21/64

(52) U.S. Cl. CPC C12Q 1/6841; G01N 21/6428; G01N 2021/6439

(57) ABSTRACT

The present disclosure generally relates to methods and compositions for in situ analysis or detection of analytes in a sample. More specifically, the present disclosure relates to methods for reducing autofluorescence in tissue samples, methods for analyzing biological samples, and compounds for use in the same. The methods and compounds of the present disclosure may be especially suitable for analytical methods employing fluorescence in situ hybridization techniques over multiple cycles of imaging.

### CROSS-REFERENCE TO RELATED APPLICATIONS

This application claims priority to U.S. Provisional Patent Application No. 63/393,022, filed Jul. 28, 2022, entitled “TADF EMITTERS FOR IN SITU DETECTION AND REDUCTION OF AUTOFLUORESCENCE,” which is herein incorporated by reference in its entirety for all purposes.

### FIELD

The present disclosure generally relates to methods, systems, and compositions for analysis or detection of analytes in a biological sample, particularly for in situ analysis of biomolecules in a cell or tissue sample using thermally activated delayed fluorescence (TADF) materials.

### BACKGROUND

Methods are available for analyzing nucleic acids in a biological sample in situ, such as a cell or a tissue. For instance, advances in single molecule fluorescent hybridization (smFISH) have enabled nanoscale-resolution imaging of RNA in cells and tissues. However, oligonucleotide probe-based assay methods for in situ analysis may suffer from low sensitivity, specificity, and/or detection efficiency, particularly in the presence of background autofluorescence. Autofluorescence is typically generated by biological moieties endogenous to the tissue and cell samples. Autofluorescence can also arise from and/or be exacerbated by standard sample treatments, such as formalin-fixation. Background autofluorescence may have a significant, ongoing impact on the ability to detect and resolve fluorescence signals from analytes of interest over other components also present in the biological samples, especially when analysis is carried out over multiple rounds of imaging.

### BRIEF SUMMARY

In some aspects, the present disclosure relates to methods for analyzing tissue samples, methods for analyzing biological samples comprising cells, and compounds and systems for use in the same. In some aspects, the methods, systems, and compounds of the present disclosure are suitable for analytical methods employing fluorescence imaging over multiple cycles and/or using multiple wavelengths.

The present disclosure includes improved systems and methods for multicolor, multi-cycle fluorescence imaging by providing methods of reducing autofluorescence when analyzing biological and/or tissue samples. The systems and methods of the present disclosure can achieve temporal resolution of background autofluorescence over multiple cycles of imaging by employing fluorescent emitters with fluorescent lifetimes greater than the fluorescent lifetime of at least one endogenous biological moiety exhibiting autofluorescence. The temporal removal of autofluorescence can eliminate the need for chemical methods of removing autofluorescence, including quenchers.

In some embodiments, provided herein is a method of detecting an analyte in a biological sample, the method comprising contacting the biological sample with a nucleic acid probe conjugated to a thermally activated delayed fluorescence (TADF) emitter, wherein the nucleic acid probe directly or indirectly binds to the analyte or a product or complex thereof in the biological sample. In some embodiments, the method comprises exciting the biological sample with a pulsed light source such that the biological sample emits autofluorescence and the TADF emitter emits fluorescence. In some embodiments, the method comprises detecting the fluorescence emitted by the TADF emitter at a location in the biological sample after a time period post excitation. In some embodiments, no or substantially no autofluorescence emitted from the biological sample is detected after the time period post excitation.

In any of the embodiments herein, the time period post excitation can be greater than the lifetime of the autofluorescence emitted from the biological sample. In any of the embodiments herein, the fluorescence emitted by the TADF emitter can be detectable before and/or within the time period post excitation. In any of the embodiments herein, the method may but does not need to comprise detecting the fluorescence emitted by the TADF emitter before and/or within the time period post excitation. In any of the embodiments herein, the time period post excitation can be less than the lifetime of the fluorescence emitted by the TADF emitter.

In any of the embodiments herein, the biological sample can be pulsed with excitation light from the pulsed light source. In any of the embodiments herein, the fluorescence emitted by the TADF emitter can be detected after a time period post each excitation pulse. In some embodiments, no or substantially no autofluorescence emitted from the biological sample is detected after the time period post each excitation pulse. In any of the embodiments herein, the method may but does not need to comprise detecting the fluorescence emitted by the TADF emitter before and/or within the time period post each excitation pulse.

In any of the embodiments herein, the end point of the time period can be at least 10 nanoseconds post excitation. In any of the embodiments herein, the end point of the time period can be at least 25 nanoseconds post excitation. In any of the embodiments herein, the end point of the time period can be at least 50 nanoseconds post excitation. In any of the embodiments herein, the end point of the time period can be at least 100 nanoseconds post excitation.

In any of the embodiments herein, the TADF emitter can comprise an electron donor and an electron acceptor. In any of the embodiments herein, the TADF emitter can comprise at least one electron donor which is a carbazole, phenoxazine, diphenylamine, triphenylamine, phenothiazine, diphenylacridine, phenazine, spiroacridine, acridine, or dimethylacridine moiety. In some embodiments, each of the aforementioned moiety can be substituted with C₁-C₆ alkyl or C₁-C₆ alkoxy. In any of the embodiments herein, the TADF emitter can comprise two, three, four, or more electron donors.

In any of the embodiments herein, the TADF emitter can comprise at least one electron acceptor which is a cyanobenzene, dicyanobenzene, diphenyltriazine, diphenylsulfone, naphthalimide, heptazine, triazine, dicyanoimidazole, curcuminoid, oxadiazole, benzothiadiazole, pyrimidine, phenylbenzimidazole, dibenzodipyridophenazine, triarylboron, or dicyanopyrazino phenanthrene moiety. In some embodiments, each of the aforementioned moiety can be substituted with C₁-C₆ alkyl or C₁-C₆ alkoxy. In any of the embodiments herein, the TADF emitter can comprise two, three, four, or more electron acceptors.

In any of the embodiments herein, the TADF emitter may but does not need to be encapsulated in and/or attached to a polymer matrix. In any of the embodiments herein, the TADF emitter may but does not need to be encapsulated in and/or attached to a nanoparticle. For example, the TADF emitters are not loaded onto polystyrene nanoparticles.

In any of the embodiments herein, the TADF emitter may be covalently or noncovalently conjugated to the nucleic acid probe. In some embodiments, the TADF emitter is covalently conjugated to the nucleic acid probe. In some embodiments, the TADF emitter is noncovalently conjugated to the nucleic acid probe.

In any of the embodiments herein, the method can further comprise functionalizing the TADF emitter prior to conjugating it to the nucleic acid probe. In any of the embodiments herein, the TADF emitter can be directly conjugated to the nucleic acid probe through a bond or indirectly conjugated to the nucleic acid probe through a linker. In some embodiments, the linker is an optionally substituted C₁-C₆ alkyl linker. In some embodiments, the linker is a polyethylene glycol linker.

In any of the embodiments herein, the detection of the fluorescence emitted by the TADF emitter can be performed with the biological sample in a buffer comprising an oxygen scavenger. In some embodiments, the buffer comprises at least one enzymatic oxygen scavenger. In any of the embodiments herein, the buffer can comprise protocatechuic acid (PCA) and protocatechuate dioxygenase (PCD). In any of the embodiments herein, the buffer can comprise lactate oxyrase, glucose oxidase or catalase, glucose pyranose oxidase, or a combination thereof. In any of the embodiments herein, the buffer can comprise at least one non-enzymatic oxygen scavenger. In any of the embodiments herein, the buffer can comprise sodium sulfite. In any of the embodiments herein, the buffer can comprise at least one protective agent. In any of the embodiments herein, the buffer can comprise Trolox, MV/AA, NAC/MV, propyl gallate, or a combination thereof.

In any of the embodiments herein, a solution or a suspension comprising the nucleic acid probe conjugated to the TADF emitter can be contacted with the biological sample. In any of the embodiments herein, the nucleic acid probe can hybridize to the analyte or product thereof. In any of the embodiments herein, the nucleic acid probe can be conjugated to a binding moiety that directly or indirectly binds to the analyte or product thereof. In some embodiments, the binding moiety comprises an antibody or epitope binding fragment thereof.

In any of the embodiments herein, the analyte can comprise a cellular nucleic acid. In some embodiments, the cellular nucleic acid is genomic DNA, mRNA, or cDNA. In some embodiments, the nucleic acid probe is a primary probe that hybridizes to the cellular nucleic acid. In some embodiments, the nucleic acid probe hybridizes to a primary probe or a product or complex thereof, wherein the primary probe hybridizes to the cellular nucleic acid. In some embodiments, the product or complex of the primary probe is selected from the group consisting of: a rolling circle amplification (RCA) product, a complex comprising an initiator and an amplifier for hybridization chain reaction (HCR), a complex comprising an initiator and an amplifier for linear oligonucleotide hybridization chain reaction (LO-HCR), a primer exchange reaction (PER) product, and a complex comprising a pre-amplifier and an amplifier for branched DNA (bDNA). In any of the embodiments herein, the nucleic acid probe can hybridize to a barcode sequence in the primary probe or product or complex thereof.

In any of the embodiments herein, the primary probe can be selected from the group consisting of: a primary probe comprising a 3′ or 5′ overhang upon hybridization to the cellular nucleic acid, optionally wherein the 3′ or 5′ overhang comprises one or more barcode sequences; a primary probe comprising a 3′ overhang and a 5′ overhang upon hybridization to the cellular nucleic acid, optionally wherein the 3′ overhang and the 5′ overhang each independently comprises one or more barcode sequences; a branched primary probe; a circular primary probe; a circularizable primary probe or probe set; a primary probe or probe set comprising a split hybridization region configured to hybridize to a splint, optionally wherein the split hybridization region comprises one or more barcode sequences; and a combination thereof.

In some embodiments, the nucleic acid probe hybridizes to an intermediate probe or a product or complex thereof, wherein the intermediate probe hybridizes to a primary probe or a product or complex thereof, and wherein the primary probe hybridizes to the cellular nucleic acid. In some embodiments, the product or complex of the intermediate probe and the product or complex of the primary probe are independently selected from the group consisting of: a rolling circle amplification (RCA) product, a complex comprising an initiator and an amplifier for hybridization chain reaction (HCR), a complex comprising an initiator and an amplifier for linear oligonucleotide hybridization chain reaction (LO-HCR), a primer exchange reaction (PER) product, and a complex comprising a pre-amplifier and an amplifier for branched DNA (bDNA). In any of the embodiments herein, the nucleic acid probe can hybridize to a barcode sequence in the intermediate probe or product or complex thereof. In any of the embodiments herein, the nucleic acid probe can hybridize to the intermediate probe in a region that does not hybridize to the primary probe or product or complex thereof.

In any of the embodiments herein, the intermediate probe can be selected from the group consisting of: an intermediate probe comprising a 3′ or 5′ overhang upon hybridization to the primary probe or product or complex thereof, optionally wherein the 3′ or 5′ overhang comprises one or more barcode sequences; an intermediate probe comprising a 3′ overhang and a 5′ overhang upon hybridization to the primary probe or product or complex thereof, optionally wherein the 3′ overhang and the 5′ overhang each independently comprises one or more barcode sequences; a branched intermediate probe; a circular intermediate probe; a circularizable intermediate probe or probe set; an intermediate probe or probe set comprising a split hybridization region configured to hybridize to a splint, optionally wherein the split hybridization region comprises one or more barcode sequences; and a combination thereof.

In any of the embodiments herein, the analyte can comprise a non-nucleic acid moiety. In some embodiments, the non-nucleic acid moiety is a protein, a carbohydrate, a lipid, a small molecule, or a complex thereof. In some embodiments, the biological sample is contacted with a labeling agent comprising: i) an analyte-binding region that directly or indirectly binds to the non-nucleic acid moiety, and ii) a reporter oligonucleotide. In some embodiments, the analyte-binding region is an antibody or epitope binding fragment thereof.

In some embodiments, the nucleic acid probe hybridizes to the reporter oligonucleotide or a product or complex thereof. In some embodiments, the nucleic acid probe hybridizes to an intermediate probe or a product or complex thereof, and wherein the intermediate probe hybridizes to the reporter oligonucleotide or a product or complex thereof. In some embodiments, the nucleic acid probe hybridizes to the intermediate probe in a region that does not hybridize to the reporter oligonucleotide or product or complex thereof. In any of the embodiments herein, the product or complex of the intermediate probe and the product or complex of the reporter oligonucleotide can be independently selected from the group consisting of: a rolling circle amplification (RCA) product, a complex comprising an initiator and an amplifier for hybridization chain reaction (HCR), a complex comprising an initiator and an amplifier for linear oligonucleotide hybridization chain reaction (LO-HCR), a primer exchange reaction (PER) product, and a complex comprising a pre-amplifier and an amplifier for branched DNA (bDNA). In any of the embodiments herein, the nucleic acid probe can hybridize to a barcode sequence in the reporter oligonucleotide or product or complex thereof or in the intermediate probe or product or complex thereof.

In any of the embodiments herein, the product or complex of the nucleic acid probe can be generated in situ in the biological sample. In any of the embodiments herein, the analyte or product or complex thereof can be detected in situ in the biological sample.

In some embodiments, provided herein is a method of detecting a plurality of analytes in a biological sample, the method comprising: (a) contacting the biological sample with a plurality of first nucleic acid probes, wherein each first nucleic acid probe is conjugated to a thermally activated delayed fluorescence (TADF) emitter and directly or indirectly binds to an analyte or a product or complex thereof in the biological sample; (b) exciting the biological sample with a pulsed light source such that the biological sample emits autofluorescence and the TADF emitters of the plurality of first nucleic acid probes emit fluorescence; and (c) detecting the fluorescence emitted by the TADF emitters at locations in the biological sample after a time period after the excitation in (b).

In some embodiments, the TADF emitters of two or more of the nucleic acid probes are the same. In any of the embodiments herein, the TADF emitters of two or more of the nucleic acid probes can be different. In any of the embodiments herein, the TADF emitters may but do not need to be encapsulated in and/or attached to a polymer matrix. In any of the embodiments herein, the TADF emitters may but do not need to be encapsulated in and/or attached to a nanoparticle.

In any of the embodiments herein, the time period post excitation can be greater than the lifetime of the autofluorescence emitted from the biological sample and less than the lifetime of the fluorescence emitted by the TADF emitters.

In any of the embodiments herein, the method can further comprise: (a′) contacting the biological sample with a plurality of second nucleic acid probes, wherein each second nucleic acid probe is conjugated to a TADF emitter and directly or indirectly binds to an analyte or a product or complex thereof in the biological sample; (b′) exciting the biological sample with a pulsed light source such that the biological sample emits autofluorescence and the TADF emitters of the plurality of second nucleic acid probes emit fluorescence; and (c′) detecting the fluorescence emitted by the TADF emitters at locations in the biological sample after a time period after the excitation in (b′).

In some embodiments, a particular first nucleic acid probe and a particular second nucleic acid probe target the same analyte or product or complex thereof. In some embodiments, the TADF emitters in the particular first nucleic acid probe and the particular second nucleic acid probe are the same. In some embodiments, the TADF emitters in the particular first nucleic acid probe and the particular second nucleic acid probe are different.

In any of the embodiments herein, the method provided herein can comprise extinguishing the fluorescence emitted by the TADF emitters, removing or inactivating the TADF emitters in the plurality of first nucleic acid probes, or removing the plurality of first nucleic acid probes, prior to contacting the biological sample with the plurality of second nucleic acid probes.

In any of the embodiments herein, the method can comprise pulsing the biological sample with excitation light of the same wavelength in the exciting of the biological sample and the TADF emitters of the plurality of second nucleic acid probes bound in the biological sample (e.g., of step (b) and step (b′)). In any of the embodiments herein, the method can comprise pulsing the biological sample with excitation light of a different wavelength in the exciting of the biological sample and the TADF emitters of the plurality of second nucleic acid probes bound in the biological sample (e.g., step (b) than the exciting of step (b′)). In any of the embodiments herein, the time period post excitation for the TADF emitters of the plurality of first nucleic acid probes in (b) can be the same as the time period post excitation for the TADF emitters of the plurality of second nucleic acid probes in (b′). In any of the embodiments herein, the time period post excitation for the TADF emitters of the plurality of first nucleic acid probes in (b) can be different from the time period post excitation for the TADF emitters of the plurality of second nucleic acid probes in (b′).

In any of the embodiments herein, the method can comprise generating a signal code sequence comprising signal codes corresponding to fluorescence detected at a particular location in the biological sample, wherein the signal code sequence comprises a signal code corresponding to the fluorescence detected in (c) and a signal code corresponding to the fluorescence detected in (c′). In some embodiments, the signal code sequence corresponds to a particular analyte, and detection of the signal code sequence at the particular location indicates a presence, a level, or an activity of the particular analyte at the particular location. In any of the embodiments herein, the method can comprise generating different signal code sequences at the locations in the biological sample, wherein each different signal code sequence corresponds to a different analyte, thereby detecting a plurality of different analytes at the locations in the biological sample.

In any of the embodiments herein, the biological sample can be a cell or tissue sample. In some embodiments, the biological sample is a fixed tissue sample, a formalin-fixed, paraffin-embedded (FFPE) sample, a frozen tissue sample, or a fresh tissue sample. In any of the embodiments herein, the biological sample can be a tissue slice between about 1 μm and about 50 μm in thickness. In some embodiments, the tissue slice is between about 5 μm and about 35 μm in thickness. In any of the embodiments herein, the biological sample can be from a melanoma tissue, brain tissue, lung tissue, tonsil tissue, intestinal tissue, kidney tissue, spleen tissue, liver tissue, breast tissue, or liver tissue. In any of the embodiments herein, the biological sample can be permeabilized. In some embodiments, the biological sample is fixed prior to permeabilization. In any of the embodiments herein, the biological sample can be embedded in a matrix. In some embodiments, the matrix comprises a hydrogel. In any of the embodiments herein, the biological sample can be crosslinked. In any of the embodiments herein, the biological sample can be cleared. In some embodiments, the clearing comprises contacting the biological sample with a proteinase. In any of the embodiments herein, the biological sample can be a hydrogel embedded and cleared tissue sample.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 illustrates a Jablonski diagram of select photophysical events that TADF emitters can undergo in the excited state.

FIG. 2 illustrates an example of a timescale for time-resolved fluorescence imaging in accordance with some embodiments disclosed herein. The time period post excitation can start from the excitation pulse or a time point after the excitation pulse, and can end when background fluorescence in the biological sample (e.g., due to endogenous biological moieties such as lipofuscin emitting autofluorescence) decreases to a level (which may but does not need to be zero) such that after the time period, the fluorescence emitted from the TADF emitters can be detected with little or no interference from autofluorescence.

### DETAILED DESCRIPTION

The present disclosure relates to methods for reducing autofluorescence when analyzing biological samples. More specifically, the present disclosure relates to methods, systems, and compositions for in situ analysis or detection of analytes in a sample, and systems and compounds for the same.

Analysis of biological samples by fluorescence in situ hybridization can hinge upon the ability to produce and detect fluorescence signals associated with analytes of interest or probes associated with the analytes of interest. Importantly, signals associated with these analytes or the probes directly or indirectly bound thereto should be distinguishable from and/or have a brighter fluorescence signal as compared to other non-analytes that may be autofluorescent and contribute to background noise. Tissue autofluorescence-which can be associated with various endogenous biological moieties including but not limited to lipofuscin, collagen, elastin, red blood cells, flavins, nicotinamide adenine dinucleotide (NADH), and even the extracellular matrix—is generally observed in most tissue types. Such autofluorescence can also exhibits a broad color emission, such that fluorescence signals associated with analytes of interest may be obscured across the visible light region of the electromagnetic spectrum—the primary band of wavelengths evaluated in in situ fluorescence imaging. As a result, certain wavelengths cannot be detected under steady state fluorescence without auxiliary treatments to reduce fluorescence.

In order to reduce autofluorescence, various commercial quencher compounds and compositions have been developed for application to biological samples. Existing commercially available quenchers for reducing autofluorescence are combined in quenching mixtures specifically tailored to certain tissues and/or types of autofluorescence. However, quenching efficiency often depends upon the quencher compound being utilized and the tissue type being analyzed. Indeed, no single universal quencher exists for all tissue types. Even under circumstances where background autofluorescence is substantially reduced, the occurrence of even minor occurrences of unwanted fluorescence may be detrimental to the detection of actual fluorescence signals. For example, punctuate background fluorescence signals may appear similar in size and shape to the fluorescence signals expected from rolling circle amplification products. Due to the similar size and shape of background autofluorescence to actual fluorescence signals, failure to reduce autofluorescence sufficiently may lead to higher readings of false positives.

An additional disadvantage of commercially available quenchers is that they may be only weakly physically adsorbed onto tissue samples when they are applied. As such, existing quenchers may not only require high concentrations to reduce background fluorescence to provide sufficient quenching but are also easily washed away with standard stripping agents typically used for fluorescence imaging across multiple cycles of nucleic acid probe hybridization and detection. Consequently, commercially available quenchers are applied and re-applied to a single tissue sample with each cycle of hybridization and imaging. The repeated treatments add significant burden to the analysis of a given tissue sample—lengthening assay time, increasing reagent expenditures and necessitating a certain level of complexity for automated assay performing equipment to accommodate the additional step of re-applying quenchers between imaging cycles.

The present disclosure includes improved methods and systems for reducing autofluorescence when analyzing tissue samples or other biological samples with multiplexed (multicolor) and reiterative (multiple cycle) fluorescence imaging techniques.

The section headings used herein are for organizational purposes only and are not to be construed as limiting the subject matter described.

The following description sets forth examples of methods, systems, parameters and the like. It should be recognized, however, that such description is not intended as a limitation on the scope of the present disclosure but is instead provided as a description of examples of embodiments.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

### I. Overview

As stated above, the present disclosure includes improved methods and systems for fluorescence imaging by providing methods of and systems for reducing autofluorescence when analyzing biological e.g., cell or tissue samples. In some embodiments, analytes are detected in multicolor and/or multi-cycle imaging rounds for highly multiplexed assays that comprises sequential hybridization of detectably labeled probes (e.g., nucleic acid probes). The methods and systems of the present disclosure can achieve reduction of background autofluorescence over multiple cycles of imaging by employing fluorescent emitters with fluorescent lifetimes greater than the fluorescent lifetime of at least one endogenous biological moiety exhibiting autofluorescence. In some instances, the temporal removal of autofluorescence can eliminate the need for chemical methods of removing autofluorescence, including quenchers.

Under steady state fluorescence, wherein the supply of photons from the excitation source is constant, portions of the autofluorescent biological moieties are continuously absorbing and subsequently emitting photons, as are portions of the emitters. Accordingly, while a portion of the autofluorescence biological moieties has relaxed from the excited state, another portion is in the process of relaxing and emitting photons, and yet another portion is undergoing excitation, such that there is a steady autofluorescence signal.

Some endogenous biological moieties have photoluminscent lifetimes of about 1-2 ns. When an excitation source is pulsed to provide only a brief supply of photons, often more than 90% of emission from autofluorescent biological moieties such as NADH/NAD+ for example, can end within 5 ns (Chacko et al, Cytometry Part A (2019) 95A: 56-69). In some instances, at least 90% of emission from autofluorescent biological moieties can end within 10 ns. Therefore, emissive materials with photoluminescent lifetimes longer than the lifetime of autofluorescence can be used in combination with a time-resolved fluorescence imaging (TFRI) technique to acquire fluorescence images wherein autofluorescence is reduced.

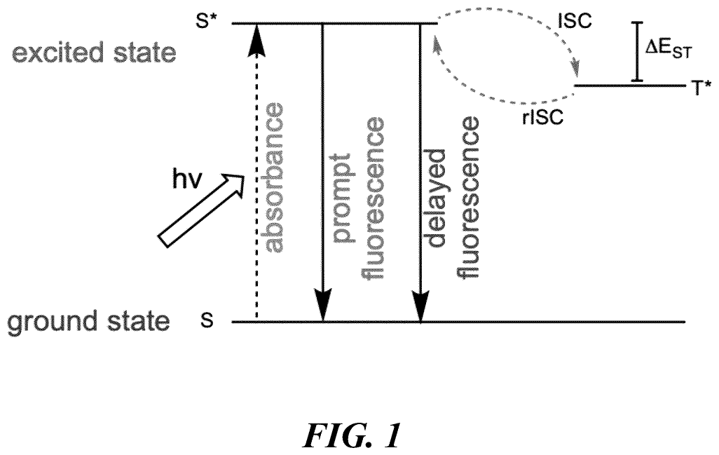

The fluorescent emitters utilized herein are referred to as thermally activated delayed fluorescence (TADF) emitters. TADF emitters can have a longer fluorescent lifetime than traditional fluorescent emitters because TADF emitters undergo additional photophysical events in the excited state. FIG. 1 depicts select photophysical events that TADF emitters can undergo in the excited state. As shown in FIG. 1, an electron is excited from the ground state (S) to the first singlet excited state (S*) upon absorption of a photon. The electron can return to S and promptly emit a photon to produce a fluorescence signal. Alternatively, the electron can transfer to the first triplet excited state (T*) through intersystem crossing, then relax to S while emitting a photon to produce a phosphorescence signal. A small energy gap between S* and T* (ΔE_{ST}) also allows for an electron in T* to undergo reverse intersystem crossing to S, where the electron can return to S and emit a photon to produce a thermally activated delayed fluorescence signal.

The photophysical pathway of traditional fluorescent emitters can include excitation followed by fluorescence from a singlet state. By contrast, the photophysical pathway of TADF emitters can include excitation followed by intersystem crossing from a singlet state to a triplet state, persistence of the triplet state for a period of time, reverse intersystem crossing from a triplet state to a singlet state, and/or fluorescent emission from the singlet state. These additional photophysical steps can contribute to an extended photoluminescent lifetime, allowing the fluorescence lifetime of the TADF emitters to persist beyond the lifetime of autofluorescence.

In some embodiments, a portion of the TADF emitters will absorb a photon and emit from the singlet state without undergoing conversion to the triplet state, resulting in prompt fluorescence with a lifetime of less than 5 ns. In some embodiments, a portion of the TADF emitters will absorb a photon, undergo intersystem crossing to the triplet state, and emit from the triplet state, resulting in phosphorescence. In some embodiments, a portion of the TADF emitters will absorb a photon and undergo non-radiative relaxation from the singlet state. In some embodiments, a portion of the TADF emitters will absorb a photon, undergo intersystem crossing to the triplet state, and undergo non-radiative relaxation from the triplet state. In some embodiments, the methods described herein comprise detecting the fluorescence signal after a period of time after excitation.

In some embodiments, the contacting of the biological sample with the nucleic acid probe conjugated to the TADF emitter (as described in Section I.B) is performed in a buffer comprising an oxygen scavenger. The triplet state of a TADF emitter can be susceptible to quenching by oxygen. In some embodiments, the buffer comprising an oxygen scavenger allows TADF emission to persist in situ. In some embodiments, the TADF emitter is not embedded or encapsulated in a polymer matrix or a nanoparticle.

A. Methods

In one aspect, provided herein are methods for reducing autofluorescence detected in analyzing a biological sample, comprising contacting a biological sample (e.g., a cell or tissue sample) with a nucleic acid probe conjugated to a thermally activated delayed fluorescence (TADF) emitter, wherein the nucleic acid probe directly or indirectly binds to the analyte or a product or complex thereof in the biological sample; exciting the biological sample with a pulsed light source such that the biological sample emits a plurality of fluorescence photons; and detecting a portion of the fluorescence photons after a time period post excitation. In some embodiments, the portion of the fluorescence photons are only detected after the time period post excitation. In some embodiments, a portion of the fluorescence photons are detected before the end point of the time period post excitation. In some embodiments, portions of fluorescence photons are detected both before and after the time period post excitation. In some instances, autofluorescence lifetimes in different tissue samples may vary and the time period post excitation can be adjusted to determine the time delay for detection.

In some embodiments, the plurality of fluorescence photons emitted from the biological sample comprise fluorescence photons emitted from autofluorescent moieties in the biological sample and fluorescence photons emitted from the TADF emitter conjugated to the nucleic acid probe. In some embodiments, after the time period post excitation, the portion of the fluorescence photons detected comprises predominantly fluorescence photons emitted from the TADF emitter and no or little autofluorescence photons emitted from the biological sample.

In some embodiments, the time period post excitation is greater than the autofluorescence lifetime of the biological sample. In some embodiments, the time period post excitation is at least about 1%, at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, or at least about 250% greater than the autofluorescence lifetime of the biological sample.

In some embodiments, the time period post excitation is less than the lifetime of the fluorescence emitted by the TADF emitter. In some embodiments, the time period post excitation is no more than about 0.01%, no more than about 0.1%, no more than about 1%, no more than about 5%, no more than about 10%, no more than about 20%, no more than about 30%, no more than about 40%, or no more than about 50% of the lifetime of the fluorescence emitted by the TADF emitter. In some embodiments, the time period post excitation is greater than the autofluorescence lifetime of the biological sample and less than the lifetime of the fluorescence emitted by the TADF emitter.

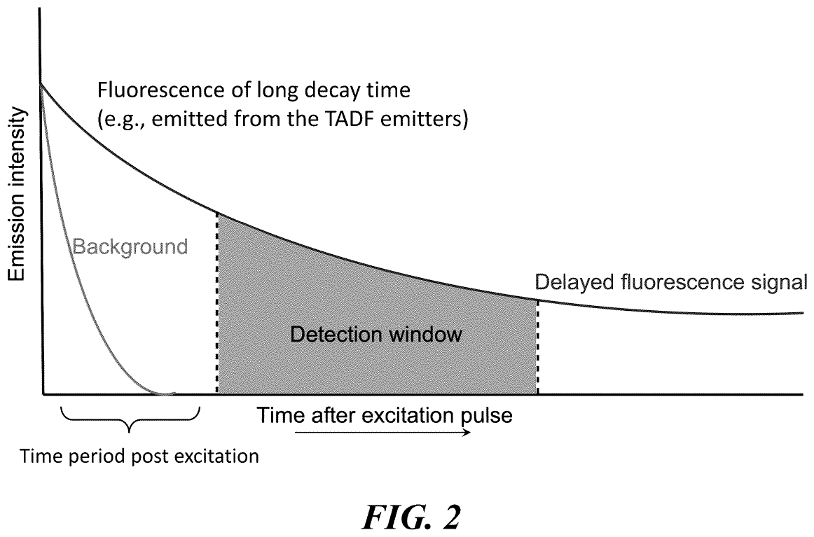

For example, FIG. 2 depicts an example of a timescale for time-resolved fluorescence imaging. As shown in FIG. 2, an excitation pulse can excite both species of molecules in the biological sample that emit background fluorescence (e.g., autofluorescence) and TADF emitters within the sample. There may be a brief period of high emission intensity due to autofluorescent biological species post excitation. The initial emission intensity quickly decreases due to the short lifetime of autofluorescence. The signal from TADF emitters (delayed fluorescent signal) can persist for a longer time, preferably outlasting the background fluorescence. A photon counting instrument can begin imaging (detection window) after autofluorescence has ended but before TADF emission has ended to produce an image without autofluorescence.

In some instances, no or substantially no autofluorescence emitted from the biological sample is detected after the time period post excitation. In some instances, at the end of the time period post excitation, the emission intensity of autofluorescence that remains detectable at that time point is no more than 75%, no more than 70%, no more than 65%, no more than 60%, more than 55%, no more than 50%, no more than 45%, no more than 40%, no more than 35%, no more than 30%, no more than 25%, no more than 20%, no more than 15%, no more than 10%, no more than 5%, no more than 1%, no more than 0.1%, or no more than 0.01% of the initial autofluorescence emission intensity, such that substantially no autofluorescence is detected during the detection of the fluorescence emitted from the TADF emitter. In some instances, the detection window for detecting fluorescence emitted from the TADF emitter begins after the time period post excitation when the background from autofluorescence is not detectable. In some instances, the background from autofluorescence is not detected within the detection window for detecting fluorescence emitted from the TADF emitter.

In some embodiments, the end point of the time period post excitation is about 1 or at least about 1 nanosecond post excitation. In some embodiments, the end point of the time period post excitation is about 5, about 10, about 15, about 20, about 25, about 30, about 35, about 40, about 45, about 50, about 55, about 60, about 65, about 70, about 75, about 80, about 85, about 90, about 95, or about 100 or more nanoseconds post excitation. In some embodiments, the end point of the time period is at least 5 nanoseconds post excitation. In some embodiments, the end point of the time period is at least 6 nanoseconds post excitation. In some embodiments, the end point of the time period is at least 7 nanoseconds post excitation. In some embodiments, the end point of the time period is at least 8 nanoseconds post excitation. In some embodiments, the end point of the time period is at least 9 nanoseconds post excitation. In some embodiments, the end point of the time period is at least about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, or between about 90 and about 100 nanoseconds post excitation. In some embodiments, the detection of fluorescence emitted from the TADF emitter does not start until about 5, about 10, about 15, about 20, about 25, about 30, about 35, about 40, about 45, about 50, about 55, about 60, about 65, about 70, about 75, about 80, about 85, about 90, about 95, or about 100 or more nanoseconds post excitation.

In some embodiments, the duration of the time period post excitation is about 5, about 10, about 15, about 20, about 25, about 30, about 35, about 40, about 45, about 50, about 55, about 60, about 65, about 70, about 75, about 80, about 85, about 90, about 95, or about 100 or more nanoseconds. In some embodiments, the duration of the time period is at least about 10, between about 10 and about 15, between about 15 and about 20, between about 20 and about 25, between about 25 and about 30, between about 30 and about 35, between about 35 and about 40, between about 40 and about 45, between about 45 and about 50, between about 50 and about 55, between about 55 and about 60, between about 60 and about 65, between about 65 and about 70, between about 70 and about 75, between about 75 and about 80, between about 80 and about 85, between about 85 and about 90, or between about 90 and about 100 nanoseconds.

In some instances, the fluorescence emitted by the TADF emitter is detected at a location in the biological sample after the end point of the time period post excitation. For example, the end point of the time period post excitation is about 5, about 10, about 15, about 20, about 25, about 30, about 35, about 40, about 45, about 50, about 55, about 60, about 65, about 70, about 75, about 80, about 85, about 90, about 95, or about 100 or more nanoseconds post excitation.

In some embodiments, post excitation, the initial emission intensity of the fluorescence emitted by the TADF emitter starts to decrease, and the detection time window for the fluorescence emitted by the TADF emitter starts when the TADF emission intensity is about 90%, about 80%, about 70%, about 60%, about 50%, about 40%, about 30%, about 20%, about 10%, or about 5% or less, of the initial TADF emission intensity. In some embodiments, at the end of the time period post excitation, the emission intensity of the fluorescence emitted by the TADF emitter is between about 90% and about 80%, between about 80% and about 70%, between about 70% and about 60%, between about 60% and about 50%, between about 50% and about 40%, between about 40% and about 30%, between about 30% and about 20%, between about 20% and about 10%, or between about 10% and about 5% of the initial emission intensity of the fluorescence emitted by the TADF emitter.

In some embodiments, the biological sample is pulsed with excitation light from the pulsed light source and the portion of fluorescence photons are detected after a time period after each excitation pulse. In some embodiments, the biological sample is pulsed with excitation light from the pulsed light source and a portion of fluorescence photons are detected before a time period after each excitation pulse. In some embodiments, the biological sample is pulsed with excitation light from the pulsed light source and portions of fluorescence photons are detected both before and after a time period after each excitation pulse. In some embodiments, the portion of fluorescence photons are only detected after the time period after each excitation pulse.

In some embodiments, the time period post excitation is at least 100 nanoseconds. In some embodiments, the time period after each excitation pulse is at least 100 nanoseconds. In some embodiments, the time period post excitation is at least 1 microsecond. In some embodiments, the time period after each excitation pulse is at least 1 microsecond.

In some embodiments, the contacting of the biological sample with the agent is performed in a buffer comprising an oxygen scavenger. In some embodiments, the buffer comprises at least one enzymatic oxygen scavenger. In some embodiments, the buffer comprises an enzyme for which the substrate is a protocatechuic acid (PCA) or a recombinant enzyme thereof. In some embodiments, the buffer comprises a protocatechuate dioxygenase (PCD), e.g., one sold by OYC Americas as bacterial protocatechuate 3,4-dioxygenase (rPCO). In some embodiments, the buffer comprises PCA and PCD. In some embodiments, the buffer comprises PCA and rPCO. In some embodiments, the buffer comprises lactate oxyrase, glucose oxidase (GOx) or catalase, glucose pyranose oxidase (P2Ox), glucose, 6-hydroxy-2,5,7,8-tetramethylchroman-2-carboxylic acid (Trolox) or a combination thereof.

In some embodiments, buffer comprises at least one non-enzymatic oxygen scavenger. In some embodiments, the buffer comprises sodium sulfite. In some embodiments, the buffer comprises at least one protective agent. In some embodiments, the buffer comprises one or more anti-blinking agents, one or more anti-bleaching agents, and/or anti-fading agents. In some embodiments, the buffer comprises one or more agents that suppress blinking of a fluorophore. In some embodiments, the buffer comprises Trolox, MV/AA, NAC/MV, propyl gallate, or a combination thereof, where MV is methyl viologen (also known as paraquat dichloride), AA is ascorbic acid, and NAC is N-acetyl-L-cysteine.

In some embodiments, prior to contacting the biological sample with one or more nucleic acid probes conjugated with TADF emitters that directly or indirectly binds to an analyte (e.g., a molecule of interest) in the biological sample, the method comprises contacting the biological sample with a probe (e.g., primary probe or intermediate probe as described in Section III) that directly or indirectly binds to the molecule of interest in the biological sample. In some embodiments, the method further comprises removing the nucleic acid probes conjugated with TADF emitters or a portion thereof from the biological sample, optionally wherein the removing step comprises treating the biological sample with a denaturing agent and/or heating.

Additional sample processing steps may be included or omitted in the methods as described herein. For example, in some embodiments, the methods of the present disclosure comprise staining the biological sample. In some embodiments, which may be combined with the preceding embodiment, the methods further comprise fixing the biological sample. In some embodiments, which may be combined with the preceding embodiments, the methods further comprise de-crosslinking the biological sample. In some embodiments, the step of contacting the biological sample with the agent may be carried out before or after one or more treatments for staining, permeabilizing, fixing and/or de-crosslinking the biological sample.

In some embodiments wherein the biological sample is a tissue sample, similar treatments and may be taken. For example, in some embodiments, the methods of the present disclosure comprise staining the tissue sample. In some embodiments, which may be combined with the preceding embodiment, the methods further comprise fixing the tissue sample. In some embodiments, which may be combined with the preceding embodiments, the methods further comprise de-crosslinking the tissue sample.

In some embodiments, the tissue sample is contacted with the nucleic acid probes conjugated with TADF emitters prior to being contacted with the buffer (e.g., imaging buffer). In other embodiments, the tissue sample is contacted with the nucleic acid probes conjugated with TADF emitters after being contacted with the buffer (e.g., imaging buffer). In yet other embodiments, the tissue sample is contacted with the nucleic acid probes conjugated with TADF emitters and the buffer (e.g., imaging buffer) simultaneously. In some embodiments, the step of contacting the tissue sample with the buffer may be carried out after one or more treatments for fixing, permeabilizing/de-crosslinking, and/or staining the biological sample. For example, in some embodiments, the methods of the present disclosure comprise staining the tissue sample. In some embodiments, which may be combined with the preceding embodiment, the methods further comprise fixing the tissue sample. In some embodiments, which may be combined with the preceding embodiments, the methods further comprise de-crosslinking the tissue sample.

It should be recognized that the order of contacting the biological or tissue sample with the buffer and the nucleic acid probes conjugated with TADF emitters may be adjusted to accommodate one or more additional sample processing steps as desired. Depending upon the class of analytes to be evaluated, certain sample processing steps may be included or omitted and the order of reagent application may change.

The methods of any of the preceding aspects may also encompass multiple cycles of imaging and the accompanying treatments for addition and/or removal of nucleic acid probes conjugated with TADF emitters. As detailed herein, the oxygen scavenging buffers of the present disclosure persist in the biological sample or tissue samples being evaluated over the course of multiple rounds of washing and stripping to exchange nucleic acid probes conjugated with TADF emitters. In some instances, the oxygen scavenging buffers of the present disclosure is reapplied to the biological sample or tissue samples being evaluated between each of the multiple rounds of removing nucleic acid probes (e.g., conjugated to a TADF emitter) and contacting with subsequent nucleic acid probes.

In some embodiments of any of the preceding aspects, the methods further comprise removing the nucleic acid probes conjugated with TADF emitters or a portion thereof from the biological sample, wherein the biological sample remains in the buffer, optionally wherein the removing step comprises treating the biological sample with a denaturing agent and/or heating. In some embodiments of any of the preceding aspects, the methods further comprise removing the nucleic acid probes conjugated with TADF emitters or a portion thereof from the tissue sample, wherein the tissue sample remains in the buffer, optionally wherein the removing step comprises treating the tissue sample with a denaturing agent and/or heating. In certain embodiments, the denaturing agent comprises dimethyl sulfoxide (DMSO), formamide, and/or an alkaline.

In some embodiments of any of the preceding aspects, the methods further comprise contacting the biological sample with one or more additional nucleic acid probes conjugated with TADF emitters, wherein each additional nucleic acid probe directly or indirectly binds to an additional molecule of interest in the biological sample, and the additional molecule of interest is the same or different from the molecule of interest in a previous step. In certain embodiments of any of the preceding aspects, the methods further comprise contacting the tissue sample with one or more additional nucleic acid probes conjugated with TADF emitters, wherein each additional nucleic acid probe directly or indirectly binds to an additional molecule of interest in the tissue sample, and the additional molecule of interest is the same or different from the molecule of interest in a previous step.

In some embodiments, the method further comprises detecting a temporal signal or signals associated with the one or more additional nucleic acid probes conjugated with TADF emitters in the biological sample, wherein the autofluorescence of the biological sample remains reduced as compared to a steady state fluorescence signal. In certain embodiments, the method further comprises detecting a temporal signal or signals associated with the one or more additional nucleic acid probes conjugated with TADF emitters in the tissue sample, wherein the autofluorescence of the tissue sample remains reduced as compared to a steady state fluorescence signal.

In other embodiments, the one or more additional nucleic acid probes conjugated with TADF emitters are contacted with the biological sample and the signal or signals (e.g., from a TADF emitter) associated with the one or more additional nucleic acid probes conjugated with TADF emitters are detected in one or more cycles, optionally wherein each cycle comprises removing the additional nucleic acid probes for the cycle from the biological sample prior to contacting the biological sample with the additional nucleic acid probes for a subsequent cycle. In still other embodiments, the one or more additional nucleic acid probes conjugated with TADF emitters are contacted with the tissue sample and the signal or signals associated with the one or more additional nucleic acid probes are detected in one or more cycles, optionally wherein each cycle comprises removing the additional nucleic acid probes for the cycle from the tissue sample prior to contacting the tissue sample with the additional nucleic acid probes conjugated with TADF emitters for a subsequent cycle.

In some embodiments of any of the foregoing aspects wherein one or more additional nucleic acid probes conjugated with TADF emitters are contacted with the biological sample and the signal or signals (e.g., from a TADF emitter) associated with the one or more additional nucleic acid probes are detected in one or more cycles the methods comprise at least two, three, four, five, six, or more cycles of contacting the biological sample with the one or more additional nucleic acid probes. In some embodiments of any of the foregoing aspects wherein one or more additional nucleic acid probes are contacted with the tissue sample and the signal or signals associated with the one or more additional nucleic acid probes conjugated with TADF emitters are detected in one or more cycles the methods comprise at least two, three, four, five, six, or more cycles of contacting the tissue sample with the one or more additional nucleic acid probes.

B. TADF Emitter and Agent Conjugate

### (i) TADF Emission Profile

The visible light region of the electromagnetic spectrum is the primary region evaluated in fluorescence measurements for in situ analysis. However, many endogenous biological moieties that are autofluorescent also fluoresce in this range of wavelengths. As detailed above, the TADF emitters of the present disclosure have fluorescent lifetimes longer than the lifetime of autofluorescence and in some cases, maintain high signal specificity over multiple cycles of imaging.

In some embodiments, the TADF emitter has an emission profile with at least one emission peak with an emission maximum between about 400 nm and 700 nm, between about 400 nm and 600 nm, between about 400 nm and 500 nm, between about 500 nm and 700 nm, between about 500 nm and 600 nm, or between about 600 nm and 700 nm, and full-width half-maximum of at least about 50 nm, at least about 75 nm or at least about 100 nm. In certain embodiments, the TADF emitter has an emission profile with at least one emission peak with an absorption maximum between about 400 nm and 700 nm and full-width half-maximum of at least about 100 nm. In some embodiments, the TADF emitter has an emission profile with at least one absorption peak with an emission maximum between about 500 and about 600 nm and full-width half-maximum of at least about 100 nm.

In some embodiments, the TADF emitter has an emission lifetime of greater than about 50 μs. In some embodiments, the TADF emitter has an emission lifetime of less than about 0.5 ms. In some embodiments, the TADF emitter has an emission lifetime of about 50 μs, about 75 μs, about 100 μs, about 125 μs, about 150 μs, about 175 μs, about 200 μs, about 225 μs, about 250 μs, about 275 μs, about 300 μs, about 325 μs, about 350 μs, about 375 μs, about 400 μs, about 425 μs, about 450 μs, about 475 μs, or about 500 μs or longer. In some embodiments, the TADF emitter has an emission lifetime of about 50 μs to about 100 μs, about 100 μs to about 250 μs, about 250 μs to about 500 μs, about 50 μs to about 250 μs, about 100 μs to about 500 μs, or about 50 μs to about 0.5 ms.

### (ii) Nucleic Acid Probes Conjugated to TADF Emitter

Provided herein are nucleic acid probes conjugated with TADF emitters. In some embodiments, the TADF emitter comprises at least one electron donor. In some embodiments, the TADF emitter comprises at least one carbazole, phenoxazine, diphenylamine, triphenylamine, phenothiazine, diphenylacridine, phenazine, spiroacridine, acridine, or dimethylacridine moiety, each of which is optionally substituted with C₁-C₆ alkyl or C₁-C₆ alkoxy. In some embodiments, the TADF emitter comprises one to four carbazole, phenoxazine, diphenylamine, triphenylamine, acridine, or dimethylacridine moieties, each of which is optionally substituted with C₁-C₆ alkyl or C₁-C₆ alkoxy.

In some embodiments, the TADF emitter comprises at least one cyanobenzene, dicyanobenzene, diphenyltriazine, diphenylsulfone, naphthalimide, heptazine, triazine, dicyanoimidazole, curcuminoid, oxadiazole, benzothiadiazole, pyrimidine, phenylbenzimidazole, dibenzodipyridophenazine, triarylboron, or dicyanopyrazino phenanthrene moiety. In some embodiments, the TADF emitter comprises an organoboron moiety.

In some embodiments, the TADF emitter comprises 2,4,5,6-Tetra(9H-carbazol-9-yl)isophthalonitrile (4CzIPN), 4-(3,6-Di-tert-butyl-9H-carbazole-9-yl)phenyl 3-(3,6-di-tert-butyl-9H-carbazole-9-yl)phenyl sulfone (DTC-DPS), 10,10′-(4,4′-Sulfonylbis(4,1-phenylene))bis(9,9-dimethyl-9,10-dihydroacridine) (DMAC-DPS), N-(4-Tert-butylphenyl)-1,8-naphthalimide-9,9-dimethyl-9,10-dihydroacridine (NAI-DMAC), or 7,10 bis(diphenylamino)dibenzo[f,h]quinoxaline-2,3-dicarbonitrile (DPA-DCPP).

In some embodiments, the TADF emitter is a multi-resonance TADF emitter. In some embodiments, the multi-resonance TADF emitter has a smaller full width half maximum than emitters with traditional donor-acceptor frameworks. In some embodiments, the smaller full width half maximum may result in higher color purity. In some embodiments, the smaller full width half maximum may result in reduced cross talk between detection channels. In some embodiments, the smaller full width half maximum may result in higher color purity and reduced cross talk between detection channels. In some embodiments, the multi-resonance TADF emitter is 5,9-diphenyl-5,9-dihydro-5,9-diaza-13b-boranaphtho[3,2,1-de]anthracene (DABNA-1).

As used herein, the term “alkyl”, by itself or as part of another substituent, means, unless otherwise stated, a straight or branched chain hydrocarbon radical, having the number of carbon atoms designated (i.e., C₁-C₆ means one to six carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. In some embodiments, the term “alkyl” may encompass C₁-C₆ alkyl, C₂-C₆ alkyl, C₃-C₆ alkyl, C₄-C₆ alkyl, C₅-C₆ alkyl, C₁-C₅ alkyl, C₂-C₅ alkyl, C₃-C₅ alkyl, C₄-C₅ alkyl, C₁-C₄ alkyl, C₂-C₄ alkyl, C₃-C₄ alkyl, C₁-C₃ alkyl, C₂-C₃ alkyl, or C₁-C₂ alkyl.

As used herein, the term “alkoxy”, by itself or as part of another substituent, means, unless otherwise stated, a straight or branched chain hydrocarbon radical having one oxygen atom, having the number of carbon atoms designated (i.e., C₁-C₆ means one to six carbons). Examples of alkoxy groups include methoxy, ethoxy, t-butoxy, and the like. In some embodiments, the term “alkoxy” may encompass C₁-C₆ alkoxy, C₂-C₆ alkoxy, C₃-C₆ alkoxy, C₄-C₆ alkoxy, C₅-C₆ alkoxy, C₁-C₅ alkoxy, C₂-C₅ alkoxy, C₃-C₅ alkoxy, C₄-C₅ alkoxy, C₁-C₄ alkoxy, C₂-C₄ alkoxy, C₃-C₄ alkoxy, C₁-C₃ alkoxy, C₂-C₃ alkoxy, or C₁-C₂ alkoxy.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined herein. It will be understood by those skilled in the art, with respect to any group containing one or more substituents, that such groups are not intended to introduce any substitution or substitution patterns that are sterically impractical, synthetically non-feasible, and/or inherently unstable. It will also be understood that where a group or moiety is optionally substituted, the disclosure includes both embodiments in which the group or moiety is substituted and embodiments in which the group or moiety is unsubstituted.

As used herein, the term “electron donor” means an electron-rich chemical moiety that donates an electron to an electron acceptor upon excitation, for example from the absorption of a photon. An electron donor may comprise one or more aryl, heteroaryl, and/or heterocyclic rings, wherein two or more aryl, heteroaryl, and/or heterocyclic rings, if present, may be fused or non-fused. An electron donor may further comprise one or more electron-rich atoms covalently bonded to two or more aryl, heteroaryl, and/or heterocyclic rings. Examples of electron donors include, but are not limited to, carbazole, phenoxazine, diphenylamine, triphenylamine, acridine, phenothiazine, diphenylacridine, phenazine, spiroacridine, and dimethylacridine moieties.

As used herein, the term “electron acceptor” means an electron-deficient chemical moiety that accepts an electron from an electron donor. An electron acceptor may comprise one or more aryl, heteroaryl, and/or heterocyclic rings, wherein two or more aryl, heteroaryl, and/or heterocyclic rings, if present, may be fused or non-fused. An electron acceptor may further comprise an electron-deficient atom bonded to one or more electron-withdrawing atoms, including but not limited to carbonyl and sulfone groups. Examples of electron acceptors include, but are not limited to, cyanobenzene, dicyanobenzene, diphenyltriazine, diphenylsulfone, naphthalimide, heptazine, triazine, dicyanoimidazole, curcuminoid, oxadiazole, benzothiadiazole, pyrimidine, phenylbenzimidazole, dibenzodipyridophenazine, triarylboron, and dicyanopyrazino phenanthrene moieties.

In some embodiments, the TADF emitter is functionalized prior to conjugating it to the nucleic acid probe. The TADF emitter can be conjugated to a nucleic acid probe either directly or indirectly. In some embodiments, the TADF emitter is conjugated to the nucleic acid probe using disuccinimidyl ester activation chemistry. In some embodiments, the TADF emitter is functionalized with an amino group. In some embodiments, the TADF emitter is functionalized with an alkyl amino group. In some embodiments, the TADF emitter is functionalized with a polyethylene glycol moiety terminating in an amino group. In some embodiments, the amino group of the functionalized TADF emitter is reacted with an excess of disuccinimidyl suberate. In some embodiments, the amino group of the functionalized TADF emitter is reacted with a first succinimidyl ester of disuccinimidyl suberate. In some embodiments, an amino group of the nucleic acid probe is reacted with a second succinimidyl ester of disuccinimidyl suberate. In some embodiments, the amino group of the functionalized TADF emitter is reacted with a first succinimidyl ester of disuccinimidyl suberate, and an amino group of the nucleic acid probe is reacted with a second succinimidyl ester within the same molecule of disuccinimidyl suberate. In some embodiments, the TADF emitter is functionalized with a polypeptide. In some embodiments, the TADF emitter is functionalized with a polynucleotide. In some embodiments, the TADF emitter is functionalized with an antibody. In some embodiments, the TADF emitter is functionalized with a polypeptide, a polynucleotide, or an antibody through an optionally substituted C₁-C₆ alkyl linker. In some embodiments, the TADF emitter is functionalized with a polypeptide, a polynucleotide, or an antibody through a polyethylene glycol linker. The TADF emitter can be conjugated to the nucleic acid probe using suitable bioconjugation methods similar to antibody oligonucleotide conjugation strategies. In some instances, the TADF emitter may be joined to the nucleic acid probe enzymatically or chemically.

C. Buffer Comprising an Oxygen Scavenger

In some embodiments, the contacting of the biological sample with the nucleic acid probe conjugated to the TADF emitter is performed in a buffer (e.g., imaging buffer) comprising an oxygen scavenger. In some embodiments, the contacting of the biological sample with the nucleic acid probe conjugated to the TADF emitter is performed prior to the addition of a buffer comprising an oxygen scavenger.

In some embodiments, preparation of the biological sample comprises a step wherein the biological sample is put in contact with an imaging buffer. In some embodiments, the imaging buffer reduces fluorophore blinking. In some embodiments, the imaging buffer reduces photo-bleaching. In some embodiments, the imaging buffer reduces fluorophore blinking and photo-bleaching. In some embodiments, the imaging buffer increases fluorophore stability. In some embodiments, the imaging buffer protects DNA during imaging. In some embodiments, the imaging buffer reduces photo-crosslinking. In some embodiments, the imaging buffer reduces fluorophore blinking, reduces photo-bleaching, increases fluorophore stability, protects DNA during imaging, reduces photo-crosslinking, or a combination thereof.

In some embodiments, the buffer comprises additional effective components. In some embodiments, the buffer comprises oxygen scavenging systems. In some embodiments, oxygen scavenging systems remove molecular oxygen and reactive oxygen species. In some embodiments, the buffer comprises at least one enzymatic oxygen scavenger. In some embodiments, the buffer comprises an enzyme for which the substrate is PCA. In some embodiments, the buffer comprises PCD or a recombinant enzyme thereof (e.g., rPCO). In some embodiments, the buffer comprises PCA and PCD. In some embodiments, the buffer comprises PCA and rPCO. In some embodiments, the buffer comprises lactate oxyrase, glucose oxidase (GOx) or catalase, glucose pyranose oxidase (P2Ox), glucose, 6-hydroxy-2,5,7,8-tetramethylchroman-2-carboxylic acid (Trolox) or a combination thereof.

In some embodiments, buffer comprises at least one non-enzymatic oxygen scavenger. In some embodiments, the buffer comprises sodium sulfite. In some embodiments, the buffer further comprises at least one protective agent. In some embodiments, the buffer comprises Trolox, MV/AA, NAC/MV, propyl gallate, or a combination thereof. In some embodiments, NAC confers strong antioxidant effects. In some embodiments, NAC reduces free radicals.

D. Endogenous Biological Moiety

In some embodiments, the endogenous biological moieties present in the biological or tissue samples as described herein are autofluorescent or are associated with or proximal to another endogenous biological moiety that is autofluorescent. In some embodiments, the endogenous biological moiety is autofluorescent. An endogenous biological moiety that is autofluorescent may also be referred to herein as “an autofluorescent moiety”.

In some embodiments, the emission lifetime of the autofluorescent moiety is less than about 1 ns. In some embodiments, the emission lifetime of the autofluorescent moiety is between about 1 ns and about 2 ns. In some embodiments, the emission lifetime of the autofluorescent moiety is between about 2 ns and about 5 ns. In some embodiments, the emission lifetime of the autofluorescent moiety is between about 5 ns and about 10 ns. In some embodiments, the emission lifetime of the autofluorescent moiety is between about 2 ns and about 10 ns.

In some embodiments, an endogenous biological moiety is selected from the group consisting of lipofuscin, collagen, elastin, red blood cells, flavins, nicotinamide adenine dinucleotide (NADH) and the extracellular matrix. In some instances, autofluorescence may arise from inherently fluorescence metabolites and other endogenous biological moieties. In some embodiments, an endogenous biological moiety is lipofuscin. In other embodiments, an endogenous biological moiety is collagen. In some embodiments, an endogenous biological moiety is elastin. In other embodiments, an endogenous biological moiety are red blood cells. In yet other embodiments, an endogenous biological moiety are flavins. In yet other embodiments, an endogenous biological moiety is nicotinamide adenine dinucleotide. In still yet other embodiments, an endogenous biological moiety is the extracellular matrix. It should be recognized that various biological samples and tissue samples may comprise one or more of the aforementioned endogenous biological moieties. It should also be recognized that certain tissue types may comprise combinations of certain endogenous biological moieties which differentiate the tissue type from other tissue types.

In some embodiments, one or more of the aforementioned endogenous biological moieties may contribute to an autofluorescence signal. In some embodiments, the autofluorescence signal may persist for less than about 1 ns. In some embodiments, the autofluorescence signal may persist for between about 1 ns and about 2 ns. In some embodiments, the autofluorescence signal may persist for between about 2 ns and about 5 ns. In some embodiments, the autofluorescence signal may persist for between about 5 ns and about 10 ns. In some embodiments, the autofluorescence signal may persist for between about 2 ns and about 10 ns. In some embodiments, the autofluorescence signal may persist for more than about 10 ns.

E. Detection

As detailed herein, the methods of the present disclosure comprise a step or multiple steps of detecting one or more signals associated with one or more nucleic acid probes conjugated with TADF emitters (e.g., as described in Section I.B). The signal associated with the nucleic acid probes conjugated with TADF emitters is a fluorescence signal, which becomes observable or becomes more readily observable after a period of time after excitation according to the methods describe herein.

In some embodiments, the analyte is a molecule of interest. In some embodiments, the molecule of interest is a nucleic acid of interest or a protein of interest. In certain embodiments wherein the molecule of interest is a nucleic acid of interest, the nucleic acid probes conjugated with TADF emitters (e.g., as described in Section I.B) hybridizes to the nucleic acid of interest. In some embodiments, the nucleic acid probes conjugated with TADF emitters hybridize to a primary probe hybridized to the nucleic acid of interest (e.g., RNA). In certain other embodiments, the nucleic acid probe hybridizes to one or more intermediate probes which directly or indirectly bind to the nucleic acid of interest. In yet other embodiments, the nucleic acid probe hybridizes to one or more intermediate probes which in turn hybridize to the nucleic acid of interest. In some embodiments, the nucleic acid probe hybridizes indirectly to the nucleic acid of interest (e.g., RNA) by hybridizing to another probe hybridized to the nucleic acid of interest or a product generated therefrom.

In other embodiments, the methods further comprise adding a primary probe prior to adding the one or more intermediate probes, wherein the intermediate probe binds to the primary probe or a product thereof. In some embodiments, the primary probe is a nucleic acid probe that directly or indirectly binds to an analyte such as a cellular or viral DNA or RNA or a product thereof. In other embodiments, the intermediate probe is a nucleic acid probe that hybridizes to the primary probe or a product thereof, wherein the primary probe is a nucleic acid probe. In some instances, the nucleic acid probes conjugated with TADF emitters hybridize to the intermediate probes.

In some embodiments, the signals associated with one or more nucleic acid probes conjugated with TADF emitters are associated with a probe that is linear, circularizable, circular, or in a branched complex of hybridized probes (e.g., various probes and amplification methods are described in Section III-V). In some embodiments, a probe that binds to the analyte directly or indirect or to a primary nucleic acid probe or a product or complex thereof directly or indirectly (e.g., an amplification product) comprises a detectable label (e.g., TADF emitter) that may be detected and associated with the analyte of interest. In some embodiments, the barcode sequences (e.g., in a probe or a product thereof) are used to combinatorially encode a plurality of analytes of interest. As such, signals associated with the nucleic acid probes conjugated with TADF emitters at particular locations in a biological sample can be used to generate distinct signal signatures through a plurality of hybridization cycles, thereby identifying the analytes at the particular locations.

In some embodiments, provided herein are methods for in situ analysis of analytes in a sample comprising detecting nucleic acid molecules, such as analyte nucleic acid molecules, detectably labeled probes, hybridization complexes, and/or rolling circle amplification (RCA) products (RCPs). Thus, in some aspects provided herein is a method for analyzing a biological sample, comprising: a) generating a nucleic acid molecule (e.g., an RCP) or complex (e.g., one that comprises an analyte and detectably labeled probe(s), such as a branched structure) in the biological sample, the nucleic acid molecule or complex comprising an identifier sequence such as a barcode sequence or analyte sequence, wherein the identifier sequence is associated with an analyte of interest and is assigned a signal code sequence; b) contacting the biological sample with a first probe (e.g., an intermediate probe such as an L-probe) and a first TADF-conjugated nucleic acid probe to generate a first complex comprising the first probe hybridized to the nucleic acid molecule or complex and the first TADF-conjugated nucleic acid probe hybridized to the first probe, wherein the first probe comprises (i) a recognition sequence (e.g., a target-binding region) complementary to the identifier sequence (e.g., barcode sequence or analyte sequence) and (ii) a first landing sequence (e.g., an overhang sequence), and wherein the first TADF-conjugated nucleic acid probe comprises a sequence complementary to the first landing sequence; c) detecting a first signal associated with the TADF emitter of the first TADF-conjugated nucleic acid probe, e.g., using time-resolved detection of TADF emitters disclosed herein, wherein the first signal corresponds to a first signal code in the signal code sequence; d) contacting the biological sample with a second probe (e.g., an intermediate probe such as L-probe) and a second TADF-conjugated nucleic acid probe to generate a second complex comprising the second probe hybridized to the nucleic acid molecule or complex and the second TADF-conjugated nucleic acid probe hybridized to the second probe, wherein the second probe comprises (i) a recognition sequence (e.g., a target-binding region) complementary to the identifier sequence (e.g., barcode sequence or analyte sequence) and (ii) a second landing sequence (e.g., an overhang sequence), and wherein the second TADF-conjugated nucleic acid probe comprises a sequence complementary to the second landing sequence; and e) detecting a second signal associated with the TADF emitter of the second TADF-conjugated nucleic acid probe, e.g., using time-resolved detection of TADF emitters disclosed herein, wherein the second signal corresponds to a second signal code in the signal code sequence, wherein the signal code sequence comprising the first signal code and the second signal code is determined at a location in the biological sample, thereby decoding the identifier sequence (e.g., barcode sequence or analyte sequence) and identifying the analyte of interest at the location in the biological sample. In some embodiments, the TADF emitter of the first TADF-conjugated nucleic acid probe and the TADF emitter of the second TADF-conjugated nucleic acid probe are the same. In some embodiments, the TADF emitters of the first TADF-conjugated nucleic acid probe and the second TADF-conjugated nucleic acid probe are different. In some embodiments, the first signal code and the second signal code are the same. In some embodiments, the first signal code and the second signal code are different.

In some embodiments, the first probe (e.g., a first intermediate probe such as a first L-probe), the second probe (e.g., a second intermediate probe such as a second L-probe), and one or more subsequent probes (e.g., subsequent intermediate probe such as subsequent L-probes) are contacted with the biological sample sequentially in a pre-determined sequence which corresponds to the signal code sequence assigned to the identifier sequence (e.g., barcode sequence or analyte sequence), wherein the one or more subsequent probes each comprises (i) a recognition sequence complementary to the identifier sequence (e.g., barcode sequence or analyte sequence) and (ii) an overhang sequence complementary to a TADF-conjugated nucleic acid probe of a pool (e.g., a universal pool across different cycles of probe hybridization) of TADF-conjugated nucleic acid probes. In some embodiments, the biological sample is contacted with the first probe before the second probe and one or more subsequent probes. In some embodiments, the biological sample is contacted with the second after the first probe and before and one or more subsequent probes. In some embodiments, the biological sample is contacted with the one or more subsequent probes after the first probe. In some embodiments, the biological sample is contacted with the one or more subsequent probes after the first probe and the second probe.

In some embodiments, the first TADF-conjugated nucleic acid probe and the second TADF-conjugated nucleic acid probe are in the pool of TADF-conjugated nucleic acid probes. A pool of TADF-conjugated nucleic acid probes may comprises at least two TADF-conjugated nucleic acid probes, and may be used for multiplexing analyses of two or more target analytes (e.g., target nucleic acids) in a biological sample. In some embodiments, the contacting in b) comprises contacting the biological sample with the universal pool of TADF-conjugated nucleic acid probes, and the contacting in d) comprises contacting the biological sample with the universal pool of TADF-conjugated nucleic acid probes. In some embodiments, the universal pool of TADF-conjugated nucleic acid probes used in the contacting in b) is the same as the universal pool of TADF-conjugated nucleic acid probes used in the contacting in d). In some embodiments, the universal pool comprises TADF-conjugated nucleic acid probes each having a TADF emitter corresponding to a different nucleic acid sequence for hybridization to a landing sequence (e.g., an overhang sequence) in a probe (e.g., an intermediate probe such as an L-probe). In some embodiments, the number of different TADF-conjugated nucleic acid probes in the universal pool is four.

In some embodiments, the one or more subsequent probes are contacted with the biological sample to determine signal codes in the signal code sequence until sufficient signal codes have been determined to decode the identifier sequence (e.g., barcode sequence or analyte sequence), thereby identifying the target analyte (e.g., target nucleic acid). In some embodiments, the method further comprises a step of removing the first probe and/or the first TADF-conjugated nucleic acid probe from the biological sample before contacting the sample with a subsequent probe and a TADF-conjugated nucleic acid probe hybridizing to the subsequent probe. In some embodiments, the method further comprises a step of removing the second probe and/or the second TADF-conjugated nucleic acid probe from the biological sample, before contacting the sample with a subsequent probe and a TADF-conjugated nucleic acid probe hybridizing to the subsequent probe. In some embodiments, the method comprises a step of removing the signal (e.g., quenching or quenching) of the TADF-conjugated nucleic acid probe in the biological sample.

In some embodiments, the method further comprises identifying multiple different target analytes present at locations (e.g., different locations) in the biological sample. In some embodiments, each different target analyte is assigned a different signal code sequence and is targeted by a circularizable probe or probe set comprising a complement of a different barcode sequence of the plurality of barcode sequences. In some embodiments, the number of different probes (e.g., L-probes that have different recognition sequences that bind to the barcode sequences) in each pool of probes is greater than the number of different TADF-conjugated nucleic acid probes in the universal pool of TADF-conjugated nucleic acid probes. In some embodiments, the number of different TADF-conjugated nucleic acid probes in the universal pool is four. In some embodiments, the number of different probes in each pool of probes (e.g., L-probes) is about 10, about 20, about 30, about 40, about 50, about 100, about 200, about 500, about 1,000, or more. In some embodiments, the number of different recognition sequences (e.g., recognition sequences that bind to the barcode sequences) of probes in each pool of probes in at least about 10, such as at least any of about 20, 30, 40, 50, 100, 200, 500, 1,000, or more.

### (i) Imaging and Detection Wavelengths

In some embodiments, the methods comprise a step of detecting a signal associated with the nucleic acid probes conjugated with TADF emitters comprises imaging at one or more wavelengths between about 400 nm and about 700 nm. In some embodiments, the one or more wavelengths are selected from the group consisting of 400-450 nm, 450-500 nm, 500-550 nm, 550-600 nm, 600-650 nm, or 650-700 nm. In some embodiments, the one or more wavelengths are selected from the group consisting of 400-425 nm, 425-450 nm, 450-475 nm, 475-500 nm, 500-525 nm, 525-550 nm, 550-575 nm, 575-600 nm, 600-625 nm, 625-650 nm, 650-675 nm, or 675-700 nm. In some embodiments, the one or more wavelengths are selected from the group consisting of 488 nm, 532 nm, 561 nm, 590 nm, 640 nm, and 647 nm.

In some aspects, the detection comprising imaging is carried out using any suitable method of microscopy for capturing optical signals, such as fluorescence and phosphorescence signals. In some aspects, the detection is carried out using two-photon microscopy, confocal microscopy, epifluorescence microscopy, intact tissue expansion microscopy, and/or CLARITY™-optimized light sheet microscopy (COLM). The methods disclosed herein may be carried out in conjunction with any suitable additional method of microscopy for collecting information about the biological sample.

In some embodiments, fluorescence microscopy is used for detection and imaging of the nucleic acid probes conjugated with TADF emitters. In some aspects, a fluorescence microscope is an optical microscope that uses fluorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, fluorescence microscopy uses pulsed layer diodes. In some embodiments, pulsed layer diodes have a 20-400 μs pulse length. In some embodiments, pulsed layer diodes have a repetition rate of less than or equal to about 80 MHz. In some embodiments, pulsed layer diodes have a wavelength between about UV range and about NIR range.

In some embodiments, fluorescence microscopy uses Ti:Si lasers. In some embodiments, Ti:Si lasers have a pulse length greater than or equal to about 80 μs. In some embodiments, Ti:Si lasers have a repetition rate of less than about 70 MHz or less than about 80 MHz. In some embodiments, Ti:Si lasers have a wavelength of about NIR range.

In some embodiments, fluorescence microscopy uses a pulsed xenon flash lamp. In some embodiments, a pulsed xenon flash lamp has a pulse length of greater than or equal to about 2 microseconds. In some embodiments, a pulsed xenon flash lamp has a repetition range of less than or equal to about 1000 Hz. In some embodiments, a pulsed xenon flash lamp has a variable wavelength. In some embodiments, a pulsed xenon flash lamp uses a monochromator to select an excitation wavelength.

In some embodiments, confocal microscopy is used for detection and imaging of the nucleic acid probes conjugated with TADF emitters. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

In some aspects, the provided methods comprise imaging the nucleic acid probe conjugated to the TADF emitter. The term “fluorophore” comprises a substance or a portion thereof that is capable of exhibiting fluorescence in the detectable range. Particular examples of labels that may be used in accordance with the provided embodiments comprise, but are not limited to 4CzIPN, DTC-DPS, DMAC-DPS, DABNA-1, NAI-DMAC, and DPA-DCPP. In some embodiments, a detectably labeled probe (e.g., nucleic acid probe conjugated to the TADF emitter) can be used to detect one or more other polynucleotide(s) and/or amplification products, e.g., described in Sections II-V.

(ii) Improvement in Background Autofluorescence, Signal-to-Noise Ratio, and/or Detectable Object Density

As described herein, the methods of the present disclosure provide improved fluorescence imaging by reducing background autofluorescence detected as compared to analytical methods using steady state emission and traditional fluorescent emitters. The improvements in imaging may be observed as reductions in background autofluorescence levels, increased signal-to-noise ratios, and even increased detectable object count densities as compared to signals collected under steady state emission.

It should also be recognized that comparison of autofluorescence background intensity, signal-to-noise ratio, detectable object count density, and/or fluorescence intensity of nucleic acid probes conjugated with TADF emitters refers to comparison of the characteristic metric at the same wavelength.

As utilized herein, the term “autofluorescence” is used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Resolution of autofluorescence may manifest or be characterized by a reduction in the average detected light intensity or detector photoelectron counts for a biological sample. In some embodiments, the temporal autofluorescence signal of the biological sample is reduced as compared to the autofluorescence signal under steady state emission. In certain embodiments, the autofluorescence is reduced by at least about 50%, at least about 60%, at least about 70%, at least about 80% or at least about 90% as compared to the autofluorescence signal under steady state emission. In some instances, autofluorescence is reduced by at least about 99%. In some embodiments, the autofluorescence of the tissue sample is reduced as compared to the autofluorescence signal under steady state emission.

In some embodiments, the combination of the reduction of the background autofluorescence at a point after excitation and the persisting signal intensity associated with the TADF emitters herein may result in improvements in signal-to-noise ratios. As referred to herein, the phrase “signal-to-noise ratio” (or SNR or S/N) is a comparison of the level of desired signal (e.g., fluorescence signal associated with nucleic acid probes conjugated with TADF emitters) to the level of background noise. In other embodiments, the signal-to-noise ratio is increased as compared to the signal collected under steady state emission. In certain embodiments, the signal-to-noise ratio is increased by at least about 10%, at least about 20%, at least about 25%, at least about 30%, at least about 35% or at least about 40% as compared to the signal collected under steady state emission. In other embodiments, the signal-to-noise ratio is increased as compared to the signal collected under steady state emission. In certain embodiments, the signal-to-noise ratio is increased by at least about 10%, at least about 20%, or at least about 25% as compared to the signal collected under steady state emission.

In other embodiments, the reduction of background autofluorescence may also allow for observation of low intensity signals, which might otherwise have been obscured by autofluorescence. In some embodiments, the reduction of background autofluorescence may allow for improved decoding of detected signals, which might otherwise have been undecodable due to background autofluorescence. This improvement in imaging may be characterized by increases in detectable object count density. As referred to herein, the phrase “detectable object count density”, “detectable object density”, or “detected object density” is a measure of the number of objects (e.g., fluorescent objects) that can be detected for a given area of a biological sample. In some embodiments, the given area of the biological sample is a nucleus area, for example, as measured by DAPI staining, and the detectable object count density is the detected object count per unit nuclei area (e.g., object count/μm² nuclei area) in cells of the biological sample. In some embodiments, the detectable object count density is increased as compared to the signal collected under steady state emission. In certain embodiments, the detectable object count density is increased by at least about 1.2 times, at least about 1.3 times, at least about 1.4 times, at least about 1.5 times, at least about 1.6 times, at least about 1.7 times, at least about 1.8 times, or at least about 1.9 times as compared to the signal collected under steady state emission. In certain embodiments, the detectable object count density is increased by at least about 2 times, at least about 3 times, at least about 4 times, or at least about 5 times as compared to the signal collected under steady state emission.

Similar to the above improvements observed for detection of signal associated with nucleic acid probes conjugated with TADF emitters in one imaging cycle, improvements to the imaging as determined by reductions in autofluorescence, increases in signal-to-noise ratios and/or increases in detectable object count density may also be measured when one or more additional probes are detected in one or more cycles. Any reductions in autofluorescence, increases in signal-to-noise ratios and/or increases in detectable object count density may be characterized relative to the signal collected under steady state emission for a tissue sample having undergone the same number of imaging cycles.

In some embodiments, after a given cycle, the biological sample remains in the buffer and autofluorescence of the biological sample remains reduced as compared to a steady state fluorescence signal. In certain embodiments, after a given cycle, the tissue sample remains in the buffer and autofluorescence of the tissue sample remains reduced as compared to a steady state fluorescence signal.

In some embodiments, after a given cycle, the biological sample remains in the buffer and autofluorescence of the biological sample remains reduced as compared to a steady state fluorescence signal. In some embodiments, the autofluorescence remains reduced by at least about 50%, at least about 60%, at least about 70% or at least about 80% for a given cycle as compared to a steady state fluorescence signal.

In some embodiments, after a given cycle, the signal-to-noise ratio remains increased as compared to a steady state fluorescence signal. In some embodiments, the signal-to-noise ratio remains increased by at least about 10%, at least about 20%, or at least about 25% for a given cycle as compared to a steady state fluorescence signal. In some embodiments, after a given cycle, the signal-to-noise ratio remains increased as compared to a steady state fluorescence signal sample. In some embodiments, the signal-to-noise ratio remains increased by at least about 10%, at least about 20%, or at least about 25% for a given cycle as compared to a steady state fluorescence signal.

In some embodiments, the detectable object count density remains increased for a given cycle as compared to a steady state fluorescence signal. In certain embodiments, the detectable object count density remains increased by at least about 2 times, at least about 3 times, at least about 4 times, or at least about 5 times for a given cycle as compared to a steady state fluorescence signal. In certain other embodiments, the detectable object count density decreases by less than about less than about than about 5%, less than about 10% or less than about 15% for a given cycle as compared to the preceding cycle. In some embodiments, the detectable object count density remains increased for a given cycle as compared to a steady state fluorescence signal. In certain embodiments, the detectable object count density remains increased by at least about 2 times, at least about 3 times, at least about 4 times, or at least about 5 times for a given cycle as compared to a steady state fluorescence signal. In certain other embodiments, the detectable object count density decreases by less than about less than about than about 5%, less than about 10% or less than about 15% for a given cycle as compared to the preceding cycle.

### II. Samples, Analytes, and Target Sequences

A. Samples and Sample Processing

A sample disclosed herein can be or derived from any biological sample. In some embodiments, a sample herein is one in which analysis of analytes (e.g., target molecules) and their position in two- or three-dimensional space is desired. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be obtained as a tissue sample, such as a tissue section, biopsy, a core biopsy, needle aspirate, or fine needle aspirate. The biological sample can be obtained as a section of a cell pellet or a cell block. The biological sample can be obtained as a population of cells, for instance, dissociated cells obtained from a tissue sample or a cell culture. The dissociated cells can be deposited and/or immobilized on a substrate, for instance, for analysis using a platform for in situ detection of one or more analytes in the cells. The sample can be a fluid sample, such as a blood sample, urine sample, or saliva sample, and cells and cellular components therein may be analyzed after placing the cells or cellular components on a substrate. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface and the cells can be isolated from a bodily sample, e.g., blood, plasma, serum, urine, saliva, mucosal excretions, sputum, stool, and tears.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include target molecules (e.g., protein, RNA, and/or DNA) in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

The biological sample within the 3D matrix may be cleared of proteins and/or lipids that are not targets of interest. For example, the biological sample can be cleared of proteins (also called “deproteination”) by enzymatic proteolysis. The clearing step may be performed before or after covalent immobilization of any target molecules or derivatives thereof.

In some cases, the clearing step is performed after covalent immobilization of target molecules (e.g., RNA or DNA), primers, derivatives of target molecules (e.g., cDNA or amplicons), or probes to a synthetic 3D matrix. Performing the clearing step after immobilization can enable any subsequent nucleic acid hybridization reactions to be performed under conditions where the sample has been substantially deproteinated, as by enzymatic proteolysis (“protein clearing”). This method can have the benefit of removing ribosomes and other RNA- or nucleic-acid-target-binding proteins from the target molecule (while maintaining spatial location), where the protein component may impede or inhibit probe binding.

The clearing step can comprise removing non-targets from the 3D matrix. The clearing step can comprise degrading the non-targets. The clearing step can comprise exposing the sample to an enzyme (e.g., a protease) able to degrade a protein. The clearing step can comprise exposing the sample to a detergent.

Proteins may be cleared from the sample using enzymes, denaturants, chelating agents, chemical agents, and the like, which may break down the proteins into smaller components and/or amino acids. These smaller components may be easier to remove physically, and/or may be sufficiently small or inert such that they do not significantly affect the background. Similarly, lipids may be cleared from the sample using surfactants or the like. In some cases, one or more of these agents are used, e.g., simultaneously or sequentially. Non-limiting examples of suitable enzymes include proteinases such as proteinase K, proteases or peptidases, or digestive enzymes such as trypsin, pepsin, or chymotrypsin. Non-limiting examples of suitable denaturants include guanidine HCl, acetone, acetic acid, urea, or lithium perchlorate. Non-limiting examples of chemical agents able to denature proteins include solvents such as phenol, chloroform, guanidinium isocyananate, urea, formamide, etc. Non-limiting examples of surfactants include Triton X-100 (polyethylene glycol p-(1,1,3,3-tetramethylbutyl)-phenyl ether), SDS (sodium dodecyl sulfate), Igepal CA-630, or poloxamers. Non-limiting examples of chelating agents include ethylenediaminetetraacetic acid (EDTA), citrate, or polyaspartic acid. In some embodiments, compounds such as these may be applied to the sample to clear proteins, lipids, and/or other components. For instance, a buffer solution (e.g., containing Tris or tris(hydroxymethyl)aminomethane) may be applied to the sample, then removed.

In some cases, nucleic acids that are not target molecules of interest may also be cleared. These non-target nucleic acids may be removed with an enzyme to degrade nucleic acid molecules. Non-limiting examples of DNA enzymes that may be used to remove DNA include DNase I, dsDNase, a variety of restriction enzymes, etc. Non-limiting examples of techniques to clear RNA include RNA enzymes such as RNase A, RNase T, or RNase H, or chemical agents, e.g., via alkaline hydrolysis (for example, by increasing the pH to greater than 10). Non-limiting examples of systems to remove sugars or extracellular matrix include enzymes such as chitinase, heparinases, or other glycosylases. Non-limiting examples of systems to remove lipids include enzymes such as lipidases, chemical agents such as alcohols (e.g., methanol or ethanol), or detergents such as Triton X-100 or sodium dodecyl sulfate. In this way, the background of the sample may be removed, which may facilitate analysis of the nucleic acid probes or other targets, e.g., using fluorescence microscopy, or other techniques as described herein.

In some embodiments, a sample disclosed herein may be provided on a substrate. In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

In some embodiments of the methods provided herein, the biological sample is a tissue sample. The methods for reducing autofluorescence and analyzing a biological sample as detailed herein are especially suitable for tissue samples which contain a wide range of endogenous biological moieties that can contribute to background autofluorescence.

In some embodiments, the tissue sample is melanoma tissue, brain tissue, lung tissue, tonsil tissue, intestinal tissue, kidney tissue, spleen tissue, liver tissue, breast tissue, or liver tissue. Certain tissue types, including but not limited to brain, liver and tonsil tissue samples, are predisposed to displaying high levels of background fluorescence and may benefit from the methods as detailed herein for reducing autofluorescence and analyzing tissue samples. In certain embodiments, the tissue sample is brain tissue, liver tissue or tonsil tissue.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes. In some embodiments, one or more post-fixing step is performed after a hybridization complex comprising a probe and a target is formed in a sample.

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

Fixation reagents may induce or exacerbate autofluorescence in tissue samples. Accordingly, the methods of the present disclosure for reducing autofluorescence and analyzing a biological sample, such as a tissue sample, as provided herein are also suited for applications to samples which have been subjected to a fixation step. In some embodiments, the tissue sample is a formalin-fixed tissue sample or a formalin-fixed paraffin-embedded (FFPE) sample. In some embodiments, the autofluorescence is fixative-induced autofluorescence.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed, e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). In some aspects, the embedding material can be applied to the sample one or more times. Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation methods.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, US2016/0024555, US2019/0276881, US2020/0071751, WO2020/0076976, WO2020/0076979, and WO2020/0096687, the entire contents of which are incorporated herein by reference. Tissue or cell samples can be embedded within conductive hydrogels. U.S. Pat. Publ. No. 2011/0256183 (Frank et al.), U.S. Pat. No. 10,138,509 (Church et al.), U.S. Pat. No. 10,545,075 (Deisseroth et al.) and U.S. Pat. Publ. No. 2019/0233878 (Delaney, et al.) which are herein incorporated by reference, describe hydrogels and their use for embedding tissues and cells.

### (v) Staining

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained using any suitable methods of destaining or discoloring a biological sample, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly or irreversibly cross-linked prior to, during, or after an assay step disclosed herein. A cross-linking agent includes a chemical agent, or even light, that facilitates the attachment of one molecule to another molecule. Any suitable cross-linking agents including, protein-nucleic acid cross-linking agents, nucleic acid-nucleic acid cross-linking agents, protein-protein cross-linking agents may be used. In some embodiments, a cross-linking agent is a reversible cross-linking agent. In some embodiments, a cross-linking agent is a non-reversible cross-linking agent.

In some embodiments, the sample to be analyzed is contacted with a protein-nucleic acid cross-linking agent, a nucleic acid-nucleic acid cross-linking agent, a protein-protein cross-linking agent or any combination thereof. In some examples, a cross-linker is a reversible cross-linker, such that the cross-linked molecules can be easily separated. In some examples, a cross-linker is a non-reversible cross-linker, such that the cross-linked molecules cannot be easily separated. In some examples, a cross-linker is light, such as UV light. In some examples, a cross linker is light activated. These cross-linkers include formaldehyde, disuccinimidyl glutarate, UV-254, psoralens and their derivatives such as aminomethyltrioxsalen, glutaraldehyde, ethylene glycol bis[succinimidylsuccinate], and other compounds, including those described in the Thermo Scientific Pierce Cross-linking Technical Handbook, Thermo Scientific (2009) as available on the world wide web at piercenet.com/files/1601673_Cross-link_HB_Intl.pdf.

In some embodiments, target molecules can be present within a three dimensional matrix material and covalently attached to the three dimensional matrix material such that the relative position of each target molecule is fixed, e.g., immobilized, within the three dimensional matrix material. In this manner, a three-dimensional matrix of covalently bound target molecules of any desired sequence is provided. Each target molecule has its own three dimensional coordinates within the matrix material and each target molecule represents information. In this manner, a large amount of information can be stored in a three dimensional matrix.

In some embodiments, a cross-linkable probe is used to anchor target molecules to a three dimensional matrix such that the relative position of each target molecule is fixed. In embodiments, the sample is contacted with a poly-dT anchor probe to bind and anchor polyadenylated (polyA) RNAs to the matrix. In some embodiments, the anchor probe (e.g., the poly-dT anchor probe) comprises a terminal acrydite moiety or other crosslinkable moiety, which can be covalently incorporated into the matrix (e.g., during matrix polymerization). In some embodiments, the poly-dT anchor probe can be about 10 to 20 nucleotides in length (e.g., about 15-nucleotides in length). In some embodiments, the anchor probe can comprise locked-nucleic acid bases to stabilize the hybridization of the poly-dT anchor probe to polyA tails of the RNAs.

According to a further aspect, the target molecules can be amplified products of an analyte, such as amplicons produced within the three dimensional matrix material. The amplicons can then be covalently attached to the matrix, for example, by copolymerization or cross-linking. This results in a structurally stable and chemically stable three dimensional matrix of target molecules. According to this aspect, the three dimensional matrix of target molecules allows for prolonged information storage and read-out cycles. Furthermore, the position of the target molecules in the sample can be stable.

In some aspects, the analytes, polynucleotides and/or amplification product (e.g., amplicon) of an analyte or a probe bound thereto can be anchored to a polymer matrix. For example, the polymer matrix can be a hydrogel. In some embodiments, the polymer matrix comprises functional moieties. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, crosslinking chemistry may be used to anchor functional moieties of the one or more of the polynucleotide probe(s) and/or amplification product (e.g., amplicon) to other molecules and/or to the polymer matrix. For example, any suitable functional moieties can be used, such as an amine, acrydite, alkyne, biotin, azide, and thiol. In some embodiments for crosslinking, the functional moiety may be cross-linked to modified dNTP or dUTP or both. In some cases, a combination of anchoring approaches (e.g., functional moieties) can be used, e.g., to anchor one or more types of molecules to the polymer matrix.

In some embodiments, the anchoring may comprise using an acrylamide group or click chemistry moiety. In some aspects, one or more of the polynucleotide probe(s) and/or amplification product (e.g., amplicon) thereof can comprise modified nucleotides that may have the functional group directly (e.g., acrylamide, click chemistry) or be further modified (e.g., amine modified with an NHS ester chemistry) to contain the functional group. In some embodiments, click reaction chemistry may be used to couple one or more of the target molecules (or a product or derivative thereof), polynucleotide probe(s), and/or amplification product (e.g., amplicon) to the matrix (e.g., hydrogel). Any suitable click reaction and click reactive groups may be used. In some cases, a molecule may be tethered via a click reaction to a click reactive group functionalized hydrogel matrix (e.g., click gel). For example, the 5′azidomethyl-dUTP can be incorporated into a product or derivative of the target molecule, polynucleotide probe(s), and/or amplification product (e.g., amplicon) and then immobilized to the hydrogel matrix functionalized with alkyne groups. In some embodiments, a buffer can be used for click reaction catalyzation, e.g., a Cu(I)-catalyzed alkyne-azide cycloaddition (abbreviated as CuAAC) click reaction catalyzing buffer, which catalyzes the alkyne-azide bond in the click reaction.

In some embodiments, a product or derivative of the target molecule, polynucleotide probe(s), and/or amplification product (e.g., amplicon) may be functionalized by adding nucleotide triphosphate analogs comprising functional moieties for immobilization. In some examples, the nucleotide triphosphate analogs include, but are not limited to, amino-allyl dUTP, 5-TCO-PEG4-dUTP, C8-Alkyne-dUTP, 5-Azidomethyl-dUTP, 5-Vinyl-dUTP, 5-Ethynyl dUTP, and other nucleotide triphosphate analogs comprising a functional moiety for immobilization by cross-linking, or forming a chemical bond between the molecule and the matrix.

In some embodiments, the matrix comprises a cellular or synthetic matrix that contains chemical moieties (e.g., reactive groups) that can react with the functional moieties in the product or derivative of the target molecule, polynucleotide probe(s), and/or amplification product (e.g., amplicon) through functionalization reactions. For example, amino-allyl dUTP may be cross-linked to endogenous free amine groups present in proteins and other biomolecules present within the endogenous or exogenous cellular matrix, or present in a modified synthetic hydrogel matrix, such as an amine-functionalized polyacrylamide hydrogel formed by copolymerization of polyacrylamide and N-(3-aminopropyl)-methacrylamide. In some cases, nucleoside analogs containing azide functional moieties may be cross-linked to a synthetic hydrogel matrix comprising alkyne functional moieties, such as that formed by copolymerization of acrylamide and propargyl acrylamide.

In some embodiments, the biological sample is immobilized in a hydrogel via cross-linking of the polymer material that forms the hydrogel. Cross-linking can be performed chemically and/or photochemically, or alternatively by any other suitable hydrogel-formation methods. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, transposase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of analytes out of the sample, and/or to facilitate transfer of species (such as probes) into the sample. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by any suitable non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to opening up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V.A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some embodiments, any of the target nucleic acid molecules described herein can correspond to an analyte. For instance, a target nucleic acid molecule can be an endogenous nucleic acid analyte (e.g., DNA or RNA), a product of an endogenous nucleic acid analyte, a probe that directly or indirectly binds to an endogenous nucleic acid analyte, or a product of a probe that directly or indirectly binds to an endogenous nucleic acid analyte. In some aspects, a target analyte disclosed herein may similarly include any analyte of interest. In some examples, a target or analyte can be directly or indirectly detected. In some embodiments, a nucleic acid probe conjugated to a thermally activated delayed fluorescence (TADF) emitter as described herein (e.g., in Section I) binds directly or indirectly binds to the analyte or a product or complex thereof.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template. Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes or Molecules of Interest

In some embodiments, a target molecule herein corresponds to an analyte that is endogenous to a biological sample and can include nucleic acid analytes and non-nucleic acid analytes. In some embodiments, the endogenous analytes as detailed herein may also be referred to as analytes of interest or molecules of interest. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labelling agent that comprises a reporter oligonucleotide and binds directly or indirectly to a non-nucleic acid analyte) in any suitable combination. The nucleic acid probe or probe set (e.g., primary probe), the reporter oligonucleotide, or a product (e.g., hybridization or amplification product) thereof can be detected using one or more nucleic acid probes each conjugated to a TADF emitter as described in Section I.B.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Examples of analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, a biological analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence. In some embodiments, the analyte comprises one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

In some embodiments, the analyte is a nucleic acid of interest or a protein of interest. In some embodiments, the molecule of interest is a nucleic acid of interest. In some embodiments, the nucleic acid of interest is a cellular or viral DNA or RNA or a product thereof. In certain embodiments, the cellular or viral DNA or RNA or product thereof is a genomic DNA, a coding RNA, a non-coding RNA, or a cDNA. In certain other embodiments, the nucleic acid of interest is an mRNA.

In other embodiments, the analyte is a protein of interest. In some embodiments, the nucleic acid of interest is a reporter oligonucleotide in a labelling agent comprising a binding moiety that directly or indirectly binds to a non-nucleic acid analyte, optionally wherein the reporter oligonucleotide comprises one or more barcode regions.

### (ii) Labelling Agents

In some embodiments, provided herein are methods and compositions for analyzing endogenous analytes (e.g., RNA, ssDNA, and cell surface or intracellular proteins and/or metabolites) in a sample using one or more labelling agents. In some embodiments, an analyte labelling agent may interact with an analyte (e.g., an endogenous analyte in a sample). In some embodiments, the labelling agents can comprise a reporter oligonucleotide that is indicative of the analyte or portion thereof interacting with the labelling agent. For example, the reporter oligonucleotide may comprise a barcode sequence that permits identification of the labelling agent. In some cases, the sample contacted by the labelling agent can be further contacted with a probe (e.g., nucleic acid probe conjugated to the TADF emitter), that hybridizes to a reporter oligonucleotide of the labelling agent, in order to identify the analyte associated with the labelling agent. In some embodiments, the analyte labelling agent comprises an analyte binding moiety and a labelling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labelling agents.

In the methods and systems described herein, one or more labelling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, a binder or an analyte binding moiety may include any molecule or moiety capable of binding to an analyte (e.g., an analyte, e.g., a macromolecular constituent). A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have coupled thereto a first reporter oligonucleotide, while a labelling agent that is specific to a different cell feature (e.g., a second cell surface feature) may have a different reporter oligonucleotide coupled thereto. For a description of examples of labelling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labelling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labelling agents are the same. In some embodiments in which the plurality of analytes includes a single species of analyte, the analyte binding moieties of the plurality of analyte labelling agents are the different (e.g., members of the plurality of analyte labelling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide.

In some aspects, these reporter oligonucleotides may comprise nucleic acid barcode sequences that permit identification of the labelling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment) using chemical conjugation techniques (e.g., Lightning-Link® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing.

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to a first oligonucleotide that is complementary (e.g., hybridizes) to a sequence of the reporter oligonucleotide.

(iii) Products of Endogenous Analyte and/or Labelling Agent

In some embodiments, a product of an endogenous analyte and/or a labelling agent in a biological sample is detected by the nucleic acid probes conjugated with the TADF emitters as described in Section I.B. In some embodiments, provided herein are methods and compositions for analyzing one or more products of an endogenous analyte and/or a labelling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product) or derivative thereof is analyzed. In some embodiments, a labelling agent (or a reporter oligonucleotide attached thereto) that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product) or derivative of a labelling agent that directly or indirectly binds to an analyte in the biological sample is analyzed.

C. Target Sequences and Barcodes

In some aspects, one or more of the target sequences (e.g., of the analytes) can be associated with one or more barcode(s), e.g., at least two, three, four, five, six, seven, eight, nine, ten, or more barcodes. A barcode can be attached to an analyte or to another moiety or structure in a reversible or irreversible manner. Barcodes can allow for identification and/or quantification of individual analytes (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences corresponding to the target molecules are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub. 20210164039, which are hereby incorporated by reference in their entirety.

### III. Polynucleotides and Hybridization Complexes

The probes may comprise any of a variety of entities that can hybridize to a nucleic acid, typically by Watson-Crick base pairing, such as DNA, RNA, LNA, PNA, etc., depending on the application. The nucleic acid probe(s) typically contains a hybridization region that is able to bind to at least a portion of a target nucleic acid, in some embodiments specifically. The nucleic acid probe may be able to bind to a specific target nucleic acid (e.g., a cellular nucleic acid such as an mRNA, or other nucleic acids as discussed herein). In some embodiments, the nucleic acid probes may be detected using a detectable label (e.g., a thermally activated delayed fluorescence (TADF) emitter), and/or by using secondary nucleic acid probes able to bind to the nucleic acid probes. In some embodiments, the nucleic acid probes are compatible with one or more biological and/or chemical reactions. For instance, a nucleic acid probe disclosed herein can serve as a template or primer for a polymerase, a template or substrate for a ligase, a substrate for a click chemistry reaction, and/or a substrate for a nuclease (e.g., endonuclease for cleavage).

In some aspects, an analyte such as any polynucleotide nucleic acid molecule (e.g., DNA molecule; RNA molecule, modified nucleic acid, etc.) can be detected, such as a polynucleotide present in a cell. In some embodiments, the analyte comprises a target nucleic acid that is a coding RNA (e.g., mRNA). The target may, in some embodiments, be a single RNA molecule. In other embodiments, the target may be at least one RNA molecule, e.g., a group of 2, 3, 4, 5, 6 or more RNA molecules. These RNA molecules may differ in molecule type, and/or may differ in sequence. In some embodiments, the target nucleic acid is, for example, a non-coding RNA (e.g., tRNA, rRNA, microRNA (miRNA), mature miRNA or immature miRNA). In some embodiments, the target nucleic acid is a splice variant of an RNA molecule (e.g., mRNA, pre-mRNA, etc.) in the context of a cell. A suitable target nucleic acid can therefore be an unspliced RNA (e.g., pre-mRNA, mRNA), a partially spliced RNA, or a fully spliced RNA, etc. Target nucleic acids of interest may be variably expressed, e.g., have a differing abundance, within a cell population, wherein the methods of the present disclosure allow profiling and comparison of the expression levels of nucleic acids, comprising but not limited to, RNA transcripts, in individual cells. A target nucleic acid can also be a DNA molecule, e.g., a denatured genomic, viral, plasmid, etc. For example, the methods can be used to detect copy number variants, e.g., in a cancer cell population in which a target nucleic acid is present at different abundance in the genome of cells in the population; a virus-infected cells to determine the virus load and kinetics, and the like.

In some aspects, the methods provided herein are used to analyze a target nucleic acid, e.g., a messenger RNA molecule. In some embodiments, the target nucleic acid is an endogenous nucleic acid present in a biological sample. In some embodiments, the target nucleic acid is present in a cell in a tissue, for instance in a tissue sample or tissue section. In some embodiments, the tissue sample is an intact tissue sample or a non-homogenized tissue sample. In some embodiments, the tissue sample is a fresh tissue sample. In some embodiments, the tissue has previously been processed, e.g., fixed, embedded, frozen, or permeabilized using any of the steps and/or protocols described in Section II. In some embodiments, the target nucleic acid is an exogenous nucleic acid contacted with a biological sample.

In some aspects, the provided embodiments can be employed for in situ detection of an analyte such as a target nucleic acid in a cell, e.g., in cells of a biological sample or a sample derived from a biological sample, such as a tissue section on a solid support, such as on a transparent slide. In some aspects, provided herein are in situ assays using microscopy as a readout, e.g., detection or determination methods involving an optical readout. In some aspects, detection or determination of a sequence of one, two, three, four, five, or more nucleotides of a target nucleic acid is performed in situ in a cell in an intact tissue. In some aspects, the detection or determination is of a sequence associated with or indicative of a target nucleic acid. In some aspects, detection or determination of a sequence is performed such that the localization of the target nucleic acid (or product or a derivative thereof associated with the target nucleic acid) in the originating sample is detected. In some embodiments, the assay comprises detecting the presence or absence of an amplification product or a portion thereof (e.g., RCA product). In some embodiments, the present disclosure provides methods for high-throughput profiling one or more single nucleotides of interest in a large number of targets in situ, such as transcripts and/or DNA loci, for detecting and/or quantifying nucleic acids in cells, tissues, organs or organisms.

In some aspects, the methods disclosed herein involve the use of one or more probes or probe sets that hybridize to a target nucleic acid, such as an RNA molecule. Examples of probes may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary. In some embodiments, a primary probe (e.g., a DNA probe that directly binds to an RNA target) is amplified through rolling circle amplification. In some embodiments, the primary probes, such as a padlock probe or a probe set that comprises a circular or circularizable probe, contain one or more barcodes. In some embodiments, the primary probes, such as a padlock probe or a probe set that comprises a padlock probe, contain one or more barcodes. In some embodiments, one or more barcodes are indicative of a sequence in the target nucleic acid, such as a single nucleotide of interest (e.g., SNPs or point mutations), a dinucleotide sequence, or a short sequence of about 5 nucleotides in length. In some embodiments, any of the examples of probes described herein or products thereof can be detected using nucleic acid probes conjugated with TADF emitters.

In some embodiments, provided herein are methods for assessing one or more target nucleic acids, such as a plurality of different mRNAs, in a biological sample, such as a cell or a tissue sample (such as a tissue section). In some embodiments, the target nucleic acid comprises DNA. In some embodiments, the target nucleic acid comprises RNA. In some embodiments, the probe comprises DNA. In some embodiments, the target nucleic acid is RNA and the probe comprises DNA.

In some aspects, the provided methods are employed for in situ analysis of target analytes (e.g., nucleic acids), for example for in situ detection or multiplexed analysis in intact tissues or a sample with preserved cellular or tissue structure. In some aspects, the provided methods can be used to detect or determine the identity or amount in situ of single nucleotides of interest in target nucleic acids, for instance of single nucleotide polymorphisms of genes of interest.

In some aspects, the provided methods involve a step of contacting, or hybridizing, one or more polynucleotides, such as any of the nucleic acid probes described herein, to a cell or a sample containing a target nucleic acid with a region (e.g., single nucleotide) of interest in order to form a hybridization complex. In some aspects, the provided methods comprise one or more steps of ligating the polynucleotides, for instance of ligating the ends of a padlock probe to form a circularized probe. In some aspects, the provided methods involve a step of amplifying one of the polynucleotides (e.g., a padlock probe or a circularized probe produced therefrom), to generate an amplification product. In some aspects, the provided methods involve a step of detecting and/or determining the sequence of all or a portion of the amplification product (for example, of one or more barcodes contained in the amplification product) and/or one or more of the polynucleotides with or without amplification, for instance any barcodes contained therein. In some aspects, the provided methods involve performing one or more of the steps described herein, simultaneously and/or sequentially.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc. In some embodiments, the one or more barcode sequences individually are between about 5 and about 50 nucleotides in length.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of nucleic acid probes is less than the number of distinct targets (e.g., nucleic acid analytes and/or protein analytes) of the nucleic acid probes, and yet the distinct targets may still be uniquely identified from one another, e.g., by encoding a probe with a different combination of barcode sequences. However, not all possible combinations of a given set of barcode sequences need be used. For instance, each probe may contain 1, 2, 3, 4, 5, 6, 7, 8, 9, 10, 11, 12, 13, 14, 15, etc. or more barcode sequences. In some embodiments, a population of nucleic acid probes may each contain the same number of barcode sequences, although in other cases, there may be different numbers of barcode sequences present on the various probes. In some embodiments, the barcode sequences or any subset thereof in the population of nucleic acid probes can be independently and/or combinatorially detected and/or decoded.

### IV. Ligation, Extension, and Amplification

In some aspects, after formation of a hybridization complex comprising nucleic acid probes and/or probe sets described in Section III and the target nucleic acids, the assay further comprises one or more steps such as ligation, extension and/or amplification of the probe or probe set hybridized to the target nucleic acid. In some embodiments, the methods of the present disclosure include the step of performing rolling circle amplification in the presence of a target nucleic acid of interest. In some cases, a sequence of a ligation, extension, or amplification product can be detected using one or more nucleic acid probes each conjugated to a TADF emitter.

In some embodiments, the method comprises using a circular or circularizable construct hybridized to the analyte such as a target nucleic acid comprising the region of interest to generate a product (e.g., comprising a sequence of the region of interest or one or more barcode sequences associated with the target nucleic acid). In some aspects, the product is generated using RCA. In any of the embodiments herein, the method can comprise ligating the ends of a circularizable probe hybridized to the target RNA to form a circularized probe. In any of the embodiments herein, the method can further comprise generating a rolling circle amplification product of the circularized probe. In some embodiments, the RCA comprises a linear RCA. In some embodiments, the RCA comprises a branched RCA. In some embodiments, the RCA comprises a dendritic RCA. In some embodiments, the RCA comprises any combination of the foregoing. In any of the embodiments herein, the method can further comprise detecting a signal associated with the rolling circle amplification product in the biological sample. In some aspects, the ligation product or a derivative thereof (e.g., extension product) can be detected. In some cases, RCA is not performed.

In some embodiments, the circular construct is directly hybridized to the target nucleic acid. In some embodiments, the circular construct is formed from a padlock probe. In some embodiments, the circular construct is formed from a probe or probe set capable of DNA-templated ligation. See, e.g., U.S. Pat. No. 8,551,710, which is hereby incorporated by reference in its entirety. In some embodiments, the circular construct is formed from a probe or probe set capable of RNA-templated ligation. Examples of RNA-templated ligation probes and methods are described in US 2020/022424 which is incorporated herein by reference in its entirety. In some embodiments, the circular construct is formed from a specific amplification of nucleic acids via intramolecular ligation (e.g., SNAIL) probe set. See, e.g., U.S. Pat. Pub. 20190055594, which is hereby incorporated by reference in its entirety. In some embodiments, the circular construct is formed from a probe capable of proximity ligation, for instance a proximity ligation assay for RNA (e.g., PLAYR) probe set. See, e.g., U.S. Pat. Pub. 20160108458, which is hereby incorporated by reference in its entirety.

In some embodiments, the circular construct is indirectly hybridized to the target nucleic acid. In some embodiments, the circular construct is formed from a probe set capable of proximity ligation, for instance a proximity ligation in situ hybridization (PLISH) probe set.

In some embodiments, the circularizing step may comprise ligation selected from the group consisting of enzymatic ligation, chemical ligation, template dependent ligation, and/or template independent ligation. In any of the embodiments herein, the ligation can comprise using a ligase having an RNA-templated DNA ligase activity and/or an RNA-templated RNA ligase activity. In any of the embodiments herein, the ligation can comprise using a ligase selected from the group consisting of a Chlorella virus DNA ligase (PBCV DNA ligase), a T4 RNA ligase, a T4 DNA ligase, and a single-stranded DNA (ssDNA) ligase. In any of the embodiments herein, the ligation can comprise using a PBCV-1 DNA ligase or variant or derivative thereof and/or a T4 RNA ligase 2 (T4 Rnl2) or variant or derivative thereof.

Upon addition of a DNA polymerase in the presence of appropriate dNTP precursors and other cofactors, the amplification primer is elongated by replication of multiple copies of the template (e.g., a concatemer of the template is generated). This amplification product can be detected using, e.g., the secondary and higher order probes and detection oligonucleotides described herein. In some embodiments, the sequence of the amplicon or a portion thereof, is determined or otherwise analyzed, for example by using detectably labeled probes (e.g., nucleic acid probes each conjugated to a TADF emitter) and imaging. The analysis of the amplification products can comprise sequencing by hybridization, sequencing by ligation, and/or fluorescent in situ sequencing, and/or wherein the in situ hybridization comprises sequential fluorescent in situ hybridization. In some instances, sequencing using, e.g., the secondary and higher order probes and detection oligonucleotides described herein.

In any of the embodiments herein, the method can further comprise generating the product of the circularized probe in situ in the biological sample. In any of the embodiments herein, the product can be generated using rolling circle amplification (RCA). In any of the embodiments herein, the RCA can comprise a linear RCA, a branched RCA, a dendritic RCA, or any combination thereof. In any of the embodiments herein, the product can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase LI T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof.

In any of the embodiments herein, the product can be immobilized in the biological sample. In any of the embodiments herein, the product can be crosslinked to one or more other molecules in the biological sample.

In some aspects, during the amplification step, modified nucleotides can be added to the reaction to incorporate the modified nucleotides in the amplification product (e.g., nanoball). Some examples of modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix.

Examples of modification and polymer matrix that can be employed in accordance with the provided embodiments comprise those described in, for example, WO 2014/163886, WO2014/025392WO 2017/079406, US 2016/0024555, US 2018/0251833, US2019/0276881, US2020/0071751, WO2020/0076976, WO2020/0076979, and WO2020/0096687, which are incorporated herein by reference in their entirety. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

### V. Signal Amplification and Analysis

In some embodiments, a method disclosed herein comprises detecting one or more target nucleic acids (e.g., genomic DNA, cellular RNA such as mRNA, cDNA, viral DNA or RNA, a nucleic acid probe, a reporter oligonucleotide attached to a functional moiety such as a binding moiety, or a product thereof) in a sample using a plurality of primary probes configured to hybridize to the one or more target nucleic acids, wherein each primary probe comprises (i) a target-hybridizing region configured to hybridize to a different target region in the corresponding target nucleic acid, and (ii) a barcode region. In some embodiments, the sample is contacted with a plurality of nucleic acid probes conjugated with TADF emitters, wherein each nucleic acid probe is configured to hybridize to (i) a barcode sequence in the barcode regions of the plurality of primary probes, or (ii) a complement of the barcode sequence. In some embodiments, the method further comprises detecting a signal associated with the plurality of nucleic acid probes conjugated with TADF emitters or absence thereof at one or more locations in the sample. In some embodiments, the sample is contacted with a subsequent plurality of nucleic acid probes conjugated with TADF emitters, wherein each nucleic acid probe in the subsequent plurality is configured to hybridize to (i) a subsequent barcode sequence in the barcode regions of the plurality of primary probes, or (ii) a complement of the subsequent barcode sequence. In some embodiments, the method further comprises detecting a subsequent signal associated with the subsequent plurality of nucleic acid probes conjugated with TADF emitters or absence thereof at the one or more locations in the sample. In some embodiments, the method further comprises generating a signal code sequence comprising signal codes corresponding to the signal or absence thereof and the subsequent signal or absence thereof, respectively, at the one or more locations, wherein the signal code sequence corresponds to one of the one or more analytes (e.g., target nucleic acids), thereby identifying the target nucleic acid at the one or more locations in the sample.

In some embodiments, in situ detection of one or more target nucleic acids in a sample is performed using sequential hybridization of nucleic acid probes conjugated with TADF emitters to the plurality of primary probes, and using signals associated with the sequentially hybridized nucleic acid probes to decode signal code sequences each assigned to one of the one or more target nucleic acids in the sample. Each primary probe can be selected from the group consisting of: a probe comprising a 3′ or 5′ overhang (e.g., L-shaped probes), optionally wherein the 3′ or 5′ overhang comprises one or more barcode sequences; a probe comprising a 3′ overhang and a 5′ overhang (e.g., U-shaped probes), optionally wherein the 3′ overhang and the 5′ overhang each independently comprises one or more barcode sequences; a circular probe; a circularizable probe or probe set; a probe or probe set comprising a split hybridization region configured to hybridize to a splint, optionally wherein the split hybridization region comprises one or more barcode sequences; and a combination thereof.

In some embodiments, the method comprises generating a signal code sequence at one or more locations in a sample, the signal code sequence comprising signal codes corresponding to the signals (or absence thereof) associated with nucleic acid probes conjugated with TADF emitters for in situ hybridization that are sequentially applied to the sample, wherein the signal code sequence corresponds to an analyte in the sample, thereby detecting the analyte at the one or more of the multiple locations in the sample.

In some embodiments, the present disclosure relates to the detection of nucleic acids sequences in situ using probe hybridization cycles and generation of amplified signals associated with the probes, wherein background signal is reduced and sensitivity is increased.

Examples of in situ detection methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594, the content of which is incorporated herein by reference in its entirety), hybridization chain reaction, assembly of topologically catenated DNA structures using serial rounds of chemical ligation (clampFISH), signal amplification via hairpin-mediated concatemerization (e.g., as described in US 2020/0362398, the content of which is incorporated herein by reference in its entirety), e.g., primer exchange reactions such as signal amplification by exchange reaction (SABER) or SABER with DNA-Exchange (Exchange-SABER). In some embodiments, a non-enzymatic signal amplification method may be used. In some instances, any of such amplifications products can be detected using nucleic acid probes conjugated to a TADF emitter.

In some embodiments, a method disclosed herein comprises generating rolling circle amplification (RCA) products associated with one or more target nucleic acids in a sample. In some embodiments, the RCA products are detected in situ in a sample, thereby detecting the one or more target nucleic acids. In some embodiments, each of the RCA products comprises multiple complementary copies of a barcode sequence, wherein the barcode sequence is associated with a target nucleic acid in the sample and is assigned a signal code sequence. In some embodiments, the method comprises contacting the sample with a first nucleic acid probe conjugated with a TADF emitter comprising a recognition sequence complementary to a sequence in the complementary copies of the barcode sequence. In some embodiments, the method comprises detecting a first signal or absence thereof from the first nucleic acid probe conjugated with a TADF emitter hybridized to its corresponding sequence of the complementary copies of the barcode sequence in the RCA product, wherein the first signal or absence thereof corresponds to a first signal code in the signal code sequence. In some embodiments, the method comprises contacting the sample with a subsequent nucleic acid probe conjugated with a TADF emitter comprising a recognition sequence complementary to a sequence of the complementary copies of the barcode sequence. In some embodiments, the method comprises detecting a subsequent signal or absence thereof from the subsequent nucleic acid probe conjugated with a TADF emitter hybridized to its corresponding sequence of the complementary copies of the barcode sequence in the RCA product, wherein the subsequent signal or absence thereof corresponds to a subsequent signal code in the signal code sequence. In some embodiments, the signal code sequence comprising the first signal code and the subsequent signal code is determined at a location in the sample, thereby decoding the barcode sequence and identifying the target nucleic acid at the location in the sample.

In some embodiments, the barcode sequence comprises one or more barcode positions each comprising one or more barcode subunits. In some embodiments, a barcode position in the barcode sequence partially overlaps an adjacent barcode position in the barcode sequence. In some embodiments, the nucleic acid probe conjugated with a TADF emitter and the subsequent nucleic acid probe conjugated with a TADF emitter are in a set of nucleic acid probes each comprising the same recognition sequence. In some embodiments, the first nucleic acid probe conjugated with a TADF emitter and the subsequent nucleic acid probe conjugated with a TADF emitter comprise the same sequence for binding an upstream probe (e.g., intermediate probe). In some embodiments, the first nucleic acid probe conjugated with a TADF emitter and the subsequent nucleic acid probe conjugated with a TADF emitter comprise different sequences for binding an upstream probe (e.g., intermediate probe). In some embodiments, the TADF emitter of each nucleic acid probe and the signal code sequence is a fluorophore sequence uniquely assigned to the associated analyte. In some embodiments, the nucleic acid probes conjugated with TADF emitters in the set are contacted with the sample sequentially in a pre-determined sequence which corresponds to the signal code sequence assigned to the barcode sequence. In some embodiments, the nucleic acid probes conjugated with TADF emitters in the set are contacted with the sample to determine signal codes in the signal code sequence until sufficient signal codes have been determined to decode the barcode sequence, thereby identifying the analyte.

In some embodiments, hybridization chain reaction (HCR) can be used for signal detection in situ. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101(43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28(11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be known as the “toehold region” (or “input domain”). The first HCR monomers each further comprise a sequence which is complementary to a sequence in the exposed toehold region of the second HCR monomers. This sequence of complementarity in the first HCR monomers may be known as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived.

In some embodiments, similar to HCR reactions that use hairpin monomers, linear oligo hybridization chain reaction (LO-HCR) can be used for signal detection in situ. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. Examples of methods and compositions for LO-HCR are described in US 2021/0198723, incorporated herein by reference in its entirety.

In some embodiments, detection of nucleic acids sequences in situ comprises generating an assembly for branched signal amplification. In some embodiments, the assembly complex comprises an amplifier hybridized directly or indirectly (via one or more oligonucleotides) to an analyte. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. See, e.g., US 2022/0064697 which is incorporated herein by reference. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For examples of branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some embodiments, detection of nucleic acids sequences in situ comprises a primer exchange reaction (PER). In various embodiments, a primer with a domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a sample comprising analytes, see e.g., U.S. Pat. Pub. No. US20190106733, the content of which is incorporated herein by reference in its entirety, for examples of molecules and PER reaction components.

In some embodiments, a sample is contacted with nucleic acid probes conjugated with TADF emitters for in situ detection. In some embodiments, the nucleic acid probes each conjugated to a TADF emitter can be used to detect an RCA product. In some embodiments, nucleic acid probes each conjugated to a TADF emitter can be used to detect a hybridization chain reaction (HCR) product, a primer exchange reaction (PER) product, or a branched DNA (bDNA) structure.

In some embodiments, the in situ detection herein can comprise sequencing performed in situ by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Examples of SBS methods comprise those described for example, but not limited to, US 2007/0166705, US 2006/0188901, U.S. Pat. No. 7,057,026, US 2006/0240439, US 2006/0281109, WO 05/065814, US 2005/0100900, WO 06/064199, WO07/010,251, US 2012/0270305, US 2013/0260372, and US 2013/0079232.

In some embodiments, the in situ detection herein can comprise sequential hybridization, e.g., sequencing by hybridization and/or sequential in situ fluorescence hybridization. Sequential fluorescence hybridization can involve sequential hybridization of nucleic acid probes conjugated with TADF emitters. In some embodiments, a method disclosed herein comprises sequential hybridization of the nucleic acid probes conjugated with TADF emitters disclosed herein, including detectably labeled probes (e.g., nucleic acid probes conjugated with TADF emitters) and/or probes that are not detectably labeled per se but are capable of binding (e.g., via nucleic acid hybridization) and being detected by nucleic acid probes conjugated with TADF emitters. Examples of methods comprising sequential fluorescence hybridization of detectably labeled probes are described in US 2019/0161796, US 2020/0224244, US 2022/0010358, US 2021/0340618, and WO 2021/138676, all of which are incorporated herein by reference.

In some embodiments, the in situ detection herein can comprise sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; US and U.S. Pat. No. 6,306,597.

In some embodiments, the barcodes of the probes (e.g., primary or intermediate probes) are targeted by detectably labeled detection oligonucleotides, such as nucleic acid probes each conjugated to a TADF emitter. In some embodiments, one or more decoding schemes are used to decode the signals, such as fluorescence, for sequence determination. In any of the embodiments herein, barcodes (e.g., primary and/or secondary barcode sequences) can be analyzed using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labeled probes (e.g., detection oligonucleotides). Examples of decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); U.S. Pat. No. 10,457,980 B2; US 2016/0369329 A1; WO 2018/026873 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for in situ detection. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, the analysis and/or sequence determination involves washing to remove unbound polynucleotides, thereafter revealing a fluorescent product (e.g., signal from the TADF emitters) for imaging.

### VI. Compositions, Kits, And Systems

Provided herein is a composition that comprises with a nucleic acid probe conjugated to a thermally activated delayed fluorescence (TADF) emitter, and a suitable buffer comprising an oxygen scavenger. For example, the nucleic acid probe directly or indirectly binds to the analyte or a product or complex thereof in the biological sample.

In some embodiments, disclosed herein is a composition that comprises a target nucleic acid or product thereof bound to a nucleic acid probe conjugated to a thermally activated delayed fluorescence (TADF) emitter. In some aspects, the nucleic acid probe conjugated to a thermally activated delayed fluorescence (TADF) emitter is for binding to a sequence of an amplification product containing monomeric units of a target sequence corresponding an analyte (e.g., mRNA molecule). Also provided herein are kits, for example comprising one or more polynucleotides, e.g., any described in Section I.A, and reagents for performing the methods provided herein, for example reagents required for one or more steps comprising hybridization, ligation, amplification, detection, dissociation, and/or sample preparation as described herein. In some embodiments, the kit further comprises the analyte or a product or complex thereof.

The various components of the kit may be present in separate containers or certain compatible components may be pre-combined into a single container. In some embodiments, the kits further contain instructions for using the components of the kit to practice the provided methods. In some embodiments, the nucleic acid probes each conjugated to a thermally activated delayed fluorescence (TADF) emitter are provided separately from the buffer (e.g., imaging buffer) or other nucleic acid probes (e.g., primary probes, intermediate probes).

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer.

Provided herein is a system comprising an opto-fluidic instrument, the fluidics module is configured to deliver one or more reagents (e.g., nucleic acid probes conjugated to the TADF emitter (as described in Section I.B)) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles each after a time period post excitation. In various embodiments, the captured images may be processed in real time and/or at a later time to determine the presence of the one or more target molecules in the biological sample, as well as three-dimensional position information associated with each detected analyte. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules in their naturally occurring place (i.e., in situ) within the biological sample. For example, an opto-fluidic instrument may be an in-situ analysis system used to analyze a biological sample and detect target molecules including but not limited to DNA, RNA, proteins, antibodies, and/or the like.

In some examples, an opto-fluidic instrument may also include a sample module configured to receive the sample, and an optics module including an imaging system for exciting the biological sample with a pulsed light source such that the biological sample emits a plurality of fluorescence photons; and detecting a portion of the fluorescence photons after a time period post excitation. In some embodiments, the portion of the fluorescence photons are only detected after the time period post excitation. The in-situ analysis system may also include other ancillary modules configured to facilitate the operation of the opto-fluidic instrument, such as, but not limited to, cooling systems, motion calibration systems, etc. The detection of a portion of the fluorescence photons after a time period post excitation can be implemented by any suitable means. For example, in some embodiments, the detector is active for one or more detection time intervals. In other embodiments, physical gating is used, wherein a barrier shields the detector and is removed for detection for a period of time corresponding to the time period post excitation. In some embodiments, the time-gating of the time period post excitation is defined by a user and implemented via software. In some embodiments, the time-gated detection is used to capture a signal during any suitable detection time interval, such as any the time period post excitation that coincides with the end of autofluorescence lifetimes in different tissue samples and the time period post excitation can be adjusted to determine the time delay for detection.

### VII. Applications

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. As detailed herein, the present disclosure provides methods for analysis of said biological samples by fluorescence detection from TADF emitters. The methods of the present disclosure achieve this reduction in background autofluorescence by utilizing TADF emitters with fluorescent lifetimes that outlive the lifetime of autofluorescence. By virtue of the time that passes between excitation and signal detection, the background fluorescence is substantially reduced and remains reduced over multiple cycles of imaging. In some embodiments, the methods disclosed herein may further utilize one or more quenching agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some aspects, the provided embodiments can be applied in an in situ method of analyzing nucleic acid sequences, such as an in situ transcriptomic analysis or in situ detection. In some aspects, the embodiments can be applied in an imaging or detection method for multiplexed, multi-cycle nucleic acid analysis. In some aspects, the provided embodiments can be used to identify or detect regions of interest in target nucleic acids.

In some aspects, the embodiments can be applied in investigative and/or diagnostic applications, for example, for characterization or assessment of particular cell or a tissue from a subject. Applications of the provided method can comprise biomedical research and clinical diagnostics. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VII. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

A “barcode” is a label, or identifier, that conveys or is capable of conveying information (e.g., information about an analyte in a sample or a bead). A barcode can be part of an analyte, or independent of an analyte. A barcode can be attached to an analyte. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have any suitable alternate backbone linkage. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any suitable variety of analogs of these sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Any suitable useful non-native bases that can be included in a nucleic acid or nucleotide may be used.

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

A “primer extension” refers to any method where two nucleic acid sequences become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

A “nucleic acid extension” generally involves incorporation of one or more nucleic acids (e.g., A, G, C, T, U, nucleotide analogs, or derivatives thereof) into a molecule (such as, but not limited to, a nucleic acid sequence) in a template-dependent manner, such that consecutive nucleic acids are incorporated by an enzyme (such as a polymerase or reverse transcriptase), thereby generating a newly synthesized nucleic acid molecule. For example, a primer that hybridizes to a complementary nucleic acid sequence can be used to synthesize a new nucleic acid molecule by using the complementary nucleic acid sequence as a template for nucleic acid synthesis.

A “PCR amplification” refers to the use of a polymerase chain reaction (PCR) to generate copies of genetic material, including DNA and RNA sequences. Suitable reagents and conditions for implementing PCR are described, for example, in U.S. Pat. Nos. 4,683,202, 4,683,195, 4,800,159, 4,965,188, and 5,512,462, the entire contents of each of which are incorporated herein by reference. In a typical PCR amplification, the reaction mixture includes the genetic material to be amplified, an enzyme, one or more primers that are employed in a primer extension reaction, and reagents for the reaction. The oligonucleotide primers are of sufficient length to provide for hybridization to complementary genetic material under annealing conditions. The length of the primers generally depends on the length of the amplification domains, but will typically be at least 4 bases, at least 5 bases, at least 6 bases, at least 8 bases, at least 9 bases, at least 10 base pairs (bp), at least 11 bp, at least 12 bp, at least 13 bp, at least 14 bp, at least 15 bp, at least 16 bp, at least 17 bp, at least 18 bp, at least 19 bp, at least 20 bp, at least 25 bp, at least 30 bp, at least 35 bp, and can be as long as 40 bp or longer, where the length of the primers will generally range from 18 to 50 bp. The genetic material can be contacted with a single primer or a set of two primers (forward and reverse primers), depending upon whether primer extension, linear or exponential amplification of the genetic material is desired.

In some embodiments, the PCR amplification process uses a DNA polymerase enzyme. The DNA polymerase activity can be provided by one or more distinct DNA polymerase enzymes. In certain embodiments, the DNA polymerase enzyme is from a bacterium, e.g., the DNA polymerase enzyme is a bacterial DNA polymerase enzyme. For instance, the DNA polymerase can be from a bacterium of the genus Escherichia, Bacillus, Thermophilus, or Pyrococcus.

Suitable examples of DNA polymerases that can be used include, but are not limited to: E. coli DNA polymerase I, Bsu DNA polymerase, Bst DNA polymerase, Taq DNA polymerase, VENT™ DNA polymerase, DEEPVENT™ DNA polymerase, LongAmp® Taq DNA polymerase, LongAmp® Hot Start Taq DNA polymerase, Crimson LongAmp® Taq DNA polymerase, Crimson Taq DNA polymerase, OneTaq® DNA polymerase, OneTaq® Quick-Load® DNA polymerase, Hemo KlenTaq® DNA polymerase, REDTaq® DNA polymerase, Phusion® DNA polymerase, Phusion® High-Fidelity DNA polymerase, Platinum Pfx DNA polymerase, AccuPrime Pfx DNA polymerase, Phi29 DNA polymerase, Klenow fragment, Pwo DNA polymerase, Pfu DNA polymerase, T4 DNA polymerase and T7 DNA polymerase enzymes.

The term “DNA polymerase” includes not only naturally-occurring enzymes but also all modified derivatives thereof, including also derivatives of naturally-occurring DNA polymerase enzymes. For instance, in some embodiments, the DNA polymerase can have been modified to remove 5′-3′ exonuclease activity. Sequence-modified derivatives or mutants of DNA polymerase enzymes that can be used include, but are not limited to, mutants that retain at least some of the functional, e.g. DNA polymerase activity of the wild-type sequence. Mutations can affect the activity profile of the enzymes, e.g. enhance or reduce the rate of polymerization, under different reaction conditions, e.g. temperature, template concentration, primer concentration, etc. Mutations or sequence-modifications can also affect the exonuclease activity and/or thermostability of the enzyme.

In some embodiments, PCR amplification can include reactions such as, but not limited to, a strand-displacement amplification reaction, a rolling circle amplification reaction, a ligase chain reaction, a transcription-mediated amplification reaction, an isothermal amplification reaction, and/or a loop-mediated amplification reaction.

In some embodiments, PCR amplification uses a single primer that is complementary to the 3′ tag of target DNA fragments. In some embodiments, PCR amplification uses a first and a second primer, where at least a 3′ end portion of the first primer is complementary to at least a portion of the 3′ tag of the target nucleic acid fragments, and where at least a 3′ end portion of the second primer exhibits the sequence of at least a portion of the 5′ tag of the target nucleic acid fragments. In some embodiments, a 5′ end portion of the first primer is non-complementary to the 3′ tag of the target nucleic acid fragments, and a 5′ end portion of the second primer does not exhibit the sequence of at least a portion of the 5′ tag of the target nucleic acid fragments. In some embodiments, the first primer includes a first universal sequence and/or the second primer includes a second universal sequence.

In some embodiments, the PCR amplification products can be ligated to additional sequences using a DNA ligase enzyme. The DNA ligase activity can be provided by one or more distinct DNA ligase enzymes. In some embodiments, the DNA ligase enzyme is from a bacterium, e.g., the DNA ligase enzyme is a bacterial DNA ligase enzyme. In some embodiments, the DNA ligase enzyme is from a virus (e.g., a bacteriophage). For instance, the DNA ligase can be T4 DNA ligase. Other enzymes appropriate for the ligation step include, but are not limited to, Tth DNA ligase, Taq DNA ligase, Thermococcus sp. (strain 9oN) DNA ligase (9oNTM DNA ligase, available from New England Biolabs, Ipswich, MA), and Ampligase™ (available from Epicentre Biotechnologies, Madison, WI). Derivatives, e.g. sequence-modified derivatives, and/or mutants thereof, can also be used.

In some embodiments, genetic material is amplified by reverse transcription polymerase chain reaction (RT-PCR). The desired reverse transcriptase activity can be provided by one or more distinct reverse transcriptase enzymes, suitable examples of which include, but are not limited to: M-MLV, MuLV, AMV, HIV, ArrayScript™, MultiScribe™, ThermoScript™, and SuperScript® I, IL, III, and IV enzymes. “Reverse transcriptase” includes not only naturally occurring enzymes, but all such modified derivatives thereof, including also derivatives of naturally-occurring reverse transcriptase enzymes.

In addition, reverse transcription can be performed using sequence-modified derivatives or mutants of M-MLV, MuLV, AMV, and HIV reverse transcriptase enzymes, including mutants that retain at least some of the functional, e.g. reverse transcriptase, activity of the wild-type sequence. The reverse transcriptase enzyme can be provided as part of a composition that includes other components, e.g. stabilizing components that enhance or improve the activity of the reverse transcriptase enzyme, such as RNase inhibitor(s), inhibitors of DNA-dependent DNA synthesis, e.g. actinomycin D. Many sequence-modified derivative or mutants of reverse transcriptase enzymes, e.g. M-MLV, and compositions including unmodified and modified enzymes are commercially available, e.g. ArrayScript™, MultiScribe®, ThermoScript™, and SuperScript® I, IL, III, and IV enzymes.

Certain reverse transcriptase enzymes (e.g. Avian Myeloblastosis Virus (AMV) Reverse Transcriptase and Moloney Murine Leukemia Virus (M-MuLV, MMLV) Reverse Transcriptase) can synthesize a complementary DNA strand using both RNA (cDNA synthesis) and single-stranded DNA (ssDNA) as a template. Thus, in some embodiments, the reverse transcription reaction can use an enzyme (reverse transcriptase) that is capable of using both RNA and ssDNA as the template for an extension reaction, e.g. an AMV or MMLV reverse transcriptase.

In some embodiments, the quantification of RNA and/or DNA is carried out by real-time PCR (also known as quantitative PCR or qPCR), using any suitable techniques such as but not limited to “TAQMAN™” or “SYBR”, or on capillaries (“LightCycler® Capillaries”). In some embodiments, the quantification of genetic material is determined by optical absorbance and with real-time PCR. In some embodiments, the quantification of genetic material is determined by digital PCR. In some embodiments, the genes analyzed can be compared to a reference nucleic acid extract (DNA and RNA) corresponding to the expression (mRNA) and quantity (DNA) in order to compare expression levels of the target nucleic acids.

An “antibody” is a polypeptide molecule that recognizes and binds to a complementary target antigen. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′)2 fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

The terms “detectable label,” “optical label,” and “label” are used interchangeably herein to refer to a directly or indirectly detectable moiety that is associated with (e.g., conjugated to) a molecule to be detected, e.g., a probe for in situ assay.. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. For example, detectably labelled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, detectable labels can be incorporated during nucleic acid polymerization or amplification (e.g., Cy5®-labelled nucleotides, such as Cy5®-dCTP). Any suitable detectable label can be used.

### EXAMPLES

The following examples are included for illustrative purposes only and are not intended to limit the scope of the present disclosure.

### Example 1: Method for Resolution of Autofluorescence In Situ in Biological Samples

The present example describes an example of a method for reducing autofluorescence of human and mouse tissues by using the TADF emitter 4CzIPN in combination with time-resolved fluorescence microscopy.

Formalin-fixed paraffin embedded (FFPE) human brain tissues and fresh frozen (FrF) mouse brain tissues are obtained. The samples are sectioned to 10 μm thickness and transferred to Superfrost® Plus slides. The FFPE human brain tissues are treated multiple times with Histo-Clear® and citrate buffer to remove the embedded paraffin. The FrF mouse brain samples are fixed using 3.7% formaldehyde for 5 minutes. The slides are then incubated with nucleic acid probes targeting genes of interest. The primary circularizable nucleic acid probes are allowed to hybridize overnight in hybridization buffer at 37° C. The next day, the samples are washed using wash buffers to remove excess or unbound probes. The primary circularizable probes are then circularized in the presence of ligase, RNAse inhibitor and ligation buffer for 2 hours. Subsequently, the circularized probes are amplified using a polymerase and an amplification buffer for RCA. The RCA products are detected using intermediate probes that hybridize to the barcode sequences in the RCA product. The samples are also contacted with nucleic acid probes conjugated to a TADF emitter, e.g., 4CzIPN. For each round of imaging, the sample is in a buffer comprising an oxygen scavenger, such as a buffer comprising protocatechuic acid (PCA) and protocatechuate dioxygenase (PCD). The samples are excited with a pulse of photons, and the fluorescence image is collected from 1-3 μs after excitation. Once imaged, the TADF emitter labelled probes are stripped and contacted with additional nucleic acid probes conjugated to a TADF emitter for the next imaging round. In this manner, multiple rounds of stripping, re-probing, and imaging of signals associated with the TADF emitter labelled nucleic acid probes. The buffer comprising an oxygen scavenger is re-applied for each round of imaging.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method of detecting an analyte in a biological sample, the method comprising:
contacting the biological sample with a nucleic acid probe conjugated to a thermally activated delayed fluorescence (TADF) emitter, wherein the nucleic acid probe directly or indirectly binds to the analyte or a product or complex thereof in the biological sample;
exciting the biological sample with a pulsed light source such that the biological sample emits autofluorescence and the TADF emitter emits fluorescence; and
detecting the fluorescence emitted by the TADF emitter at a location in the biological sample after a time period post excitation.

2. (canceled)

3. The method of claim 1, wherein the time period post excitation is greater than the lifetime of the autofluorescence emitted from the biological sample and less than the lifetime of the fluorescence emitted by the TADF emitter.

4.-5. (canceled)

6. The method of claim 1, wherein the biological sample is pulsed with excitation light from the pulsed light source and the fluorescence emitted by the TADF emitter is detected after a time period post each excitation pulse.

7. (canceled)

8. The method of claim 1, wherein the end point of the time period is at least 10, 25, 50, or 100 nanoseconds post excitation.

9.-12. (canceled)

13. The method of claim 1, wherein the TADF emitter comprises at least one electron donor which is a carbazole, phenoxazine, diphenylamine, triphenylamine, phenothiazine, diphenylacridine, phenazine, spiroacridine, acridine, or dimethylacridine moiety.

14. (canceled)

15. The method of claim 1, wherein the TADF emitter comprises at least one electron acceptor which is a cyanobenzene, dicyanobenzene, diphenyltriazine, diphenylsulfone, naphthalimide, heptazine, triazine, dicyanoimidazole, curcuminoid, oxadiazole, benzothiadiazole, pyrimidine, phenylbenzimidazole, dibenzodipyridophenazine, triarylboron, or dicyanopyrazino phenanthrene moiety.

16. (canceled)

17. The method of claim 1, wherein the TADF emitter is not encapsulated in and/or attached to a polymer matrix, and/or wherein the TADF emitter is not encapsulated in and/or attached to a nanoparticle.

18. The method of claim 1, wherein the TADF emitter is covalently conjugated to the nucleic acid probe.

19. (canceled)

20. The method of claim 1, wherein the TADF emitter is directly conjugated to the nucleic acid probe through a bond or indirectly conjugated to the nucleic acid probe through a linker.

21. The method of claim 1, wherein the detection of the fluorescence emitted by the TADF emitter is performed with the biological sample in a buffer comprising an oxygen scavenger.

22.-29. (canceled)

30. The method of claim 1, wherein the nucleic acid probe hybridizes to the analyte or product thereof.

31. The method of claim 1, wherein the nucleic acid probe is conjugated to a binding moiety that directly or indirectly binds to the analyte or product thereof.

32. (canceled)

33. The method of claim 1, wherein the analyte comprises an mRNA.

34. (canceled)

35. The method of claim 33, wherein the nucleic acid probe hybridizes to a primary probe or a product or complex thereof, wherein the primary probe hybridizes to the mRNA.

36. (canceled)

37. The method of claim 35, wherein the nucleic acid probe hybridizes to a barcode sequence in the primary probe or product or complex thereof.

38. (canceled)

39. The method of claim 33, wherein the nucleic acid probe hybridizes to an intermediate probe or a product or complex thereof, wherein the intermediate probe hybridizes to a primary probe or a product or complex thereof, and wherein the primary probe hybridizes to the mRNA.

40.-43. (canceled)

44. The method of claim 1, wherein the analyte comprises a protein, a carbohydrate, a lipid, a small molecule, or a complex thereof.

45.-48. (canceled)

49. The method of claim 39, wherein the product or complex of the intermediate probe is selected from the group consisting of:
a rolling circle amplification (RCA) product,
a complex comprising an initiator and an amplifier for hybridization chain reaction (HCR),
a complex comprising an initiator and an amplifier for linear oligonucleotide hybridization chain reaction (LO-HCR),
a primer exchange reaction (PER) product, and
a complex comprising a pre-amplifier and an amplifier for branched DNA (bDNA).

50. (canceled)

51. The method of claim 1, wherein the product of the nucleic acid probe is generated and/or detected in situ in the biological sample.

52.-70. (canceled)

71. The method of claim 1, wherein the biological sample is a cell or tissue sample.

72.-77. (canceled)
